(19) United States
(12) Patent Application Publication
(10) Pub. No.: US 2026/0036521 A1
(43) Pub. Date: Feb. 5, 2026

(54) CYCLODEXTRIN FUNCTIONALIZED WAVEGUIDE SENSING LAYER COMPOSITIONS AND RELATED METHODS

(71) Applicant: Salvus, LLC, Valdosta, GA (US)

(72) Inventor: Scott WITTE, Valdosta, GA (US)

(21) Appl. No.: 19/285,058

(22) Filed: Jul. 30, 2025

Related U.S. Application Data
(60) Provisional application No. 63/677,011, filed on Jul. 30, 2024.

(51) Int. Cl. G01N 21/77; G01N 33/18

(52) U.S. Cl. CPC G01N 21/77; G01N 33/18; G01N 2021/7779

(57) ABSTRACT

A sensing layer composition is provided. The sensing layer composition is particularly suited to be adhered to at least one side of one or more waveguide channels in/on a waveguide chip of an interferometric system. The sensing layer composition includes at least one polymeric receptor and at least one amino silane. Methods for preparing a test sample composition for detection as well as methods of detecting and quantifying one or more analytes using an interferometric sensing system are also provided.

### CROSS-REFERENCE TO RELATED APPLICATIONS

The present application claims priority to U.S. Provisional No. 63/677,011 filed Jul. 30, 2024, the contents of which are incorporated herein in its entirety.

### BACKGROUND

Per-and polyfluoroalkyl substances (PFAS), are harmful compounds referred to as “Forever Chemicals” due to their persistence and longevity. These chemicals have been used since the 1950's in various products across many industries such as food and beverage, manufacturing, firefighting, and cosmetics. More recently, PFAS has been at the forefront of studies and regulatory efforts due to its damaging effects on human health from long-term and repeated exposure. The EPA has imposed strict regulatory limits on certain PFAS compounds in drinking water down to the 4 parts per trillion (ppt) level. This level of 4 ppt is set as an allowable level in water to reduce risks to human health, but often contaminated sites and certain products contain PFAS levels more than 1000×higher than the regulatory limit.

PFAS compounds consist of a fluorocarbon hydrophobic tail connected to a hydrophilic functional group. The hydrophilic head makes perfluorooctanoic acid (PFOA) water soluble and is available to undergo ionic interactions. The solubility of PFOA in water coupled with strong carbon-fluorine bonds leads to a stable and persistent molecule that is not easily destroyed. Use of PFAS over the last 70 years has created a major hazard for environmental and human health that can be linked to certain cancers and reproductive health.

Detection of PFAS levels continues to require expensive pieces of equipment such as LC/MS/MS in centralized laboratories that increase both the time and price for a result. The PFAS detection field lacks a reliable and rapid sensor for field application; to date sensors for PFAS detection require additional lab processes or equipment or remain far from commercial readiness. In the PFAS removal and destruction field however, more commercial products and processes have been developed including the use of resins that can bind PFOA in water to later be eluted off through an exchange mechanism and destroyed. The resins can have varying functional groups and pore sizes that can be altered for efficient target binding. The use of porous materials and cyclic oligosaccharides, such as cyclodextrin, have been useful in binding of PFAS in both the academic and industrial landscape. Cyclodextrin has specifically been of interest due to its rapid binding and regenerable properties, although it has not been utilized for rapid detection at the source.

While current mechanisms for PFAS binding and removal have made great strides in recent years, current issues in sensing remain such as sensitivity, speed of detection, and sample preparation requirements. These gaps in the market lead to the need for a field deployable sample preparation system and interferometric system with the ability to detect PFAS compounds of different chain lengths in a rapid, substantially interference free and sensitive matter that requires few steps from sample collection to result.

### SUMMARY OF THE DISCLOSURE

A sensing layer composition is provided. The sensing layer composition is particularly suited to be adhered to at least one side of one or more waveguide channels in/on a waveguide chip of an interferometric system. According to a particular embodiment, the sensing layer composition is adapted to bind one or more analytes that are detected via interferometric analysis.

According to one embodiment, the sensing layer composition includes at least one polymeric receptor. According to one embodiment, the at least one polymeric receptor includes a cyclodextrin cavity immobilized to the surface with a carboxylic acid substituted group. The at least one polymeric receptor is particularly useful in binding fluoro-containing substances such as, for example, per- and polyfluoroalkyl analytes. The binding and is particularly useful for use in interferometric analysis as binding and the resulting interferences result in specific interferometric patterns.

According to one embodiment, the polymeric receptor includes a cyclodextrin derivative chemically modified with one or more substituent groups. Exemplary substituent groups include, but are not limited to, succinic acid moieties and fluorinated alkyl chains. The one or more substituent groups enhance aqueous solubility, improve surface orientation, and promote optimal exposure of the cyclodextrin cavity toward incoming target analytes. According to one embodiment, the one or more substituent groups further improve the chemical compatibility between the cyclodextrin cavity and fluoro-containing substances by increasing local fluoro-containing substance concentration near the receptor site, thereby enhancing binding efficiency and sensitivity.

According to one embodiment, the sensing layer composition includes at least one amino silane to functionalize the at least one polymeric receptor to the surface. According to one embodiment, the at least one amino silane includes 3-aminopropyltriethoxysilane (APTES). The at least one silane is particularly suited to react with an active silicon dioxide (SiO₂) surface of a waveguide sensing surface to form a covalent bond. The at least one polymeric receptor, such as the cyclodextrin derivative, may be functionalized to the at least one amino silane through amine coupling. An active form of the at least one polymeric receptor may add to the at least one amino silane layer on the waveguide to form the complete sensing layer composition.

The concentration of amino silanes required for the initial covalently bond layer of sensing layer composition may vary. According to one embodiment, the sensing layer composition may include from about 0.1% w/w, 1% w/w, 5% w/w, 10% w/w, 15% w/w, 20% w/w, 25% w/w, 30% w/w, 35% w/w, 40% w/w, 45% w/w, 50% w/w, 55% w/w, 60% w/w, 65% w/w, 70% w/w, 75% w/w, 80% w/w, 85% w/w, 90% w/w, 95% w/w or more based on the total weight of the sensing layer composition.

According to one embodiment, the at least one polymeric receptor may be formed through the combination of a carboxylic acid substituted cyclodextrin derivative, 1-Ethyl-3-(3-dimethylaminopropyl) carbodiimide (EDC) and N-Hydroxy succinimide (NHS) with excess EDC and NHS in relation to the cyclodextrin. According to one embodiment, the concentration of the at least one polymeric receptor in the sensing layer composition can vary. According to one embodiment, the concentration of the at least one polymeric receptor in the sensing layer composition is about 0.1% w/w, 1% w/w, 5% w/w, 10% w/w, 15% w/w, 20% w/w, 25% w/w, 30% w/w, 35% w/w, 40% w/w, 45% w/w, 50% w/w, 55% w/w, 60% w/w, 65% w/w, 70% w/w, 75% w/w, 80% w/w, 85% w/w, 90% w/w, 95% w/w or more based on the total weight of the sensing layer composition.

According to one embodiment, the sensing layer composition exhibits hydrophilic properties and is particularly suited to detect analytes of interest in a test sample composition that includes a target sample and a water-based buffer. According to one embodiment, the water-based buffer may include a maximum of 5% v/v of one or more organic solvent such as, for example, ethanol or methanol. One or more analytes in the target sample may bind to the at least one polymeric receptor through a one or a combination of hydrophobic, electrostatic, size inclusion, fluorophilic, ionic fluorophilic, ionic interactions, van der Waals interactions, or any combination thereof. Such analytes include, but are not limited to, acids having carbon chains for 4 carbons up to 14 carbons. Exemplary analytes include, but are not limited to, perfluorooctanoic acid (PFOA), perfluorooctane sulfonic acid (PFOS), perfluorononanoic acid (PFNA), hexafluoropropylene oxide dimer acid (HFPO-DA, commonly known as GenX Chemicals), perfluorohexane sulfonic acid (PFHxS), perfluorobutanoic acid (PFBA), perfluorodecanoic acid (PFDA) and perfluorobutane sulfonic acid (PFBS).

According to one embodiment, the sensing layer composition is configured for use in an interferometric detection system, particularly for the selective detection of fluoro-containing analytes, including perfluoroalkyl and polyfluoroalkyl substances (PFAS). In certain embodiments, the interferometric system is an optical interferometric system wherein the sensing layer composition, upon analyte binding, induces a measurable shift in the optical signal due to changes in the local refractive index at the waveguide interface. The sensing layer composition may include at least one polymeric receptor that includes a carboxylic acid-substituted cyclodextrin derivative and at least one amino silane. The composition may be formulated to adhere to at least one surface of one or more waveguide channels on a waveguide chip, and may be designed to interact with one or more target analytes, such that the analyte presence selectively perturbs the optical properties of the sensing layer.

According to one embodiment, the polymeric receptor may be formed by covalently linking a carboxylic acid-substituted cyclodextrin derivative to an amino silane. An exemplary polymeric receptor includes, but is not limited to, 3-aminopropyltriethoxysilane (APTES) and may be coupled via EDC/NHS-mediated coupling chemistry. The resulting receptor-functionalized film may provide both structural adhesion to silicon dioxide waveguide surfaces and functional selectivity for target analytes. According to one embodiment, the polymeric receptor is 3-aminopropyltriethoxysilane (APTES) and the amino silane is a carboxylic acid-substituted cyclodextrin derivative such as succinyl β-cyclodextrin.

According to one embodiment, the sensing layer composition is especially suitable for detecting fluoro-containing substances, including but not limited to PFAS compounds such as perfluorooctanoic acid (PFOA), perfluorooctane sulfonic acid (PFOS), perfluorononanoic acid (PFNA), hexafluoropropylene oxide dimer acid (HFPO-DA, or GenX), perfluorohexane sulfonic acid (PFHxS), and perfluorobutane sulfonic acid (PFBS). Binding of these analytes to the cyclodextrin-containing polymer layer produces a change in the optical interference signal, enabling quantitative and selective analyte detection.

The sensing layer is hydrophilic and designed to exploit both hydrophobic and ionic interactions for enhanced analyte affinity. The polymeric receptor layer may be deposited via spin coating to form a continuous film and is compatible with aqueous buffer environments, including those containing up to 5% v/v ethanol or methanol. The film exhibits chemical stability for extended measurement periods and supports reversible binding, allowing for surface regeneration and reuse.

In some embodiments, the sensing layer composition includes 0.1% w/w to 50% w/w of each of the amino silane and the polymeric receptor, based on the total weight of the composition. The cyclodextrin component may include α, β or γ-cyclodextrin substituted at the primary hydroxyl position with a carboxylic acid moiety to enhance ionic binding affinity. According to one embodiment, the sensing layer composition provides a dynamic, reproducible response across PFAS concentrations from 5 parts per billion (ppb) to 500 parts per billion (ppb), with a coefficient of variation under 10%. To further improve detection accuracy, the sensing layer may be applied in a differential format across both sensing and reference waveguide channels, allowing internal correction for environmental variability and system noise.

The present disclosure also provides methods for preparing a test sample composition for detection of one or more analytes using an interferometric sensing system. The methods are particularly suitable for use in field-deployable or portable detection platforms and are designed to enable rapid, reliable, and sensitive detection of analytes, including perfluoroalkyl and polyfluoroalkyl substances (PFAS), from environmental or other complex sample matrices. In one aspect, the method includes collecting a target sample suspected of containing one or more analytes, performing a concentration step using solid-solid extraction media, solid-phase extraction (SPE), microbeads, or other suitable materials to produce a concentrated test sample, and subsequently adjusting the buffer conditions of the concentrated test sample to ensure compatibility with an interferometric sensing system. The buffer adjustment step is configured to reduce matrix interferences while maintaining analyte binding capacity with a functional sensing layer.

In certain embodiments, the extraction and concentration steps are adapted for field use and may be completed using portable equipment such as the interferometric system as provided herein with simplified handling protocols and minimal solvent requirements. The extraction and concentration method may be implemented in less than two hours from sample collection to result, allowing for rapid, on-site analyte detection. The extraction and concentration steps are compatible with interferometric systems that include waveguide chips having sensing layers that, in turn, include a polymeric receptor and an amino silane, where analyte binding induces a measurable shift in optical signal due to local refractive index changes.

In some embodiments, the method enables analyte enrichment into a dynamic detection range, for example, between 5 parts per billion and 500 parts per billion, with enhanced reproducibility and confidence in measurement results. The method of extraction and concentration steps supports reversible sensor binding, buffer compatibility with organic solvents such as ethanol or methanol (up to 5% v/v), and is validated against both standard laboratory analytical methods, such as LC-MS/MS Method 1633, and the interferometric detection platform described herein.

### BRIEF DESCRIPTION OF THE DRAWINGS

FIG. 1 illustrates a perspective view of one embodiment of a handheld interferometric system that may utilize the sensing layer composition.

FIG. 2A illustrates a front view of one embodiment of a handheld interferometric system that may utilize the sensing layer composition.

FIG. 2B illustrates a rear view of one embodiment of a handheld interferometric system that may utilize the sensing layer composition.

FIG. 3A illustrates a cross-sectional view of an interferometric chip that may be integrated into a cartridge system that may utilize the sensing layer composition.

FIG. 3B illustrates a bottom view of a flow cell wafer having a serpentine shaped detection microchannel.

FIG. 3C illustrates a top view of a chip illustrating the movement of a light signal through the chip.

FIG. 4 illustrates a side view of one embodiment of an optical assembly typically found in the handheld interferometric system of FIG. 1.

FIG. 5A illustrates a cross-sectional view of the optical assembly of FIG. 4.

FIG. 5B illustrates an alignment means according to one embodiment.

FIG. 5C illustrates an embodiment of a top view of the optical assembly and alignment means.

FIG. 6 illustrates the cross-sectional view of the optical assembly of FIG. 5A with one embodiment of a cartridge system inserted in the optical assembly.

FIG. 7 illustrates a top view of the optical assembly of FIG. 5A with one embodiment of a cartridge system inserted in the optical assembly.

FIG. 8A illustrates a view of the top surface of one embodiment of a single-use cartridge system.

FIG. 8B illustrates a view of the bottom surface of one embodiment of a single-use cartridge system.

FIG. 8C illustrates a view of the back surface of one embodiment of a single-use cartridge system.

FIG. 8D illustrates a view of the front surface of one embodiment of a single-use cartridge system.

FIG. 8E illustrates view of one side surface of one embodiment of a single-use cartridge system.

FIG. 8F illustrates a cross-section view (looking downward) of a one embodiment of a single-use cartridge system along the horizontal line of FIG. 8E.

FIG. 9A illustrates a view of the top surface of one embodiment of a multi-use cartridge system.

FIG. 9B illustrates a view of the bottom surface of one embodiment of a multi-use cartridge system.

FIG. 9C illustrates a view of the back surface of one embodiment of a multi-use cartridge system.

FIG. 9D illustrates a view of the front surface of one embodiment of a multi-use cartridge system.

FIG. 9E illustrates a side surface view of one embodiment of a multi-use cartridge system.

FIG. 9F illustrates a cross-section view (looking downward) of one embodiment of a multi-use cartridge system along the horizontal line of FIG. 9E.

FIG. 10 illustrates a perspective view of an alternative single-use cartridge system.

FIG. 11 illustrates a method of detecting and quantifying the level of analyte.

FIG. 12 a linear response curve for dynamic range of an interferometric system using a cyclodextrin polymer receptor functionalized to the surface of waveguide channels.

### DETAILED DESCRIPTION OF THE DISCLOSURE

One or more aspects and embodiments may be incorporated in a different embodiment although not specifically described. That is, all aspects and embodiments can be combined in any way or combination. When referring to the compounds disclosed herein, the following terms have the following meanings unless indicated otherwise. The following definitions are meant to clarify, but not limit, the terms defined. If a particular term used herein is not specifically defined, such term should not be considered indefinite. Rather, terms are used within their accepted meanings.

### Definitions

As used herein, the term “portable” refers to the capability of the interferometric systems described herein to be transported or otherwise carried to a target sample location for use according to the methods provided herein.

As used herein, the term “analyte” refers to a substance that is detected, identified, measured or any combination thereof by the systems provided herein. The analyte includes any solid, liquid, or gas affecting an environment of interest or that is targeted. The analyte includes, but is not limited to, fluoro-containing substances. Such fluoro-containing substances include various fluoro-containing chemical compounds that are not regulated. The analyte includes, but is not limited to, regulated fluoro-containing substances that may be regulated such as perfluoroalkyl and polyfluoroalkyl substances (PFAS). Exemplary PFAS include, but are not limited to, perfluorooctanoic acid (PFOA), perfluorooctane sulfonic acid (PFOS), perfluorononanoic acid (PFNA), hexafluoropropylene oxide dimer acid (HFPO-DA, commonly known as GenX Chemicals), perfluorohexane sulfonic acid (PFHxS), and perfluorobutane sulfonic acid (PFBS).

As used herein, the terms “sample” and “target sample” all refer to any substance that may be subject to the methods and systems provided herein. Particularly, these terms refer to any matter (animate or inanimate) where an analyte may be present and capable of being detected, quantified, monitored or a combination thereof in a batch or continuous manner. Suitable examples of targets include, but are not limited to, any animate or inanimate surface, water or water source (e.g., drinking water source), waste water, soil, food, ambient air, soil, cleaning products, fabrics, grease-resistant paper, cookware, personal products, stain-resistant coatings, aquatic animals (e.g., fish), fire retardants, bodily fluids (e.g., blood, breast milk, spinal fluid, cord blood, saliva, or amniotic fluid), agricultural sites, and landfills. Targets also include air, surfaces, fluids and mixtures thereof in or from manufacturing or processing facilities and laboratories. The target also encompasses exhaled breath.

As used herein, the term “point of use” refers to the applicability of the systems provided herein to be utilized by a user at or in a particular environment (e.g., on site).

“Optional” or “optionally” means that the subsequently described event or circumstance may or may not occur, and that the description includes instances where said event or circumstance occurs and instances where it does not.

As used herein, the term “buffer” refers to a carrier that is mixed with the target sample that includes at least one analyte. The buffer may also include one or more anti-foam compounds.

As used herein, the terms “test sample” and “test sample composition” may be used interchangeable and refer to a target sample combined with at least one buffer.

As used herein, the term “communication” refers to the movement of air, liquid, mist, fog, buffer, test sample composition, or other suitable source capable of carrying an analyte throughout or within the cartridge system. The term “communication” may also refer to the movement of electronic signals between components both internal and external to the cartridge systems described herein.

As used herein, the term “single-use” refers to the cartridge system being utilized in an interferometric system for a single test or assay before disposal (i.e., not re-used or used for a second time).

As used herein, the term “multiple-use” refers to the cartridge system being utilized for more than one test sample composition (e.g., assay) before disposal.

As used herein, the term “multiplex” refers to the cartridge system being utilized to detect multiple analytes from one target sample composition.

### Optical Interferometry Principles

The sensing layer compositions provided herein may be utilized in various interferometric systems. According to a particular embodiment, such systems include a detector that operates via ultrasensitive, optical waveguide interferometry. The waveguiding and the interferometry techniques are combined to detect, monitor and even measure small changes that occur in an optical beam along a propagation pathway. These changes can result from changes in the length of the beam's path, a change in the wavelength of the light, a change in the refractive index of the media the beam is traveling through, or any combination of these, as shown in Equation 1.

$$φ = 2 πLn / λ \qquad Equation l$$

According to Equation 1, φ is the phase change, which is directly proportional to the path length, L, and refractive index, n, and inversely proportional to the wavelength (λ) change. According to the systems and methods provided herein, the change in refractive index is used. Optical waveguides are utilized as efficient sensors for detection of refractive index change by probing near the surface region of the sample with an evanescent field. Particularly, the systems provided herein can detect small changes in an interference pattern.

According to one embodiment, the waveguide and interferometer act independently or in tandem to focus an interferometric diffraction pattern. According to one embodiment, the waveguide, interferometer, and sensor act independently or two parts in tandem, or collectively to focus an interferometric pattern with or without mirrors or other reflective or focal median. According to one embodiment, the waveguide and interferometer exhibit a coupling angle such that focus is at an optimum angle to allow the system to be compact and suited to be portable and hand-held.

### Interferometric System Overview

The interferometric systems as provided herein that may utilize the sensing layer composition are mobile (hand-held) and portable for ease of use in various environments. The interferometric systems include a weight and overall dimensions such that user may hold the entire interferometric system comfortably in one hand. According to one embodiment, the entire interferometric system is under three pounds. Thus, the present disclosure provides a lightweight, handheld and easy-to-use interferometric system that can rapidly, precisely, and accurately provide detection and quantification of analytes in a variety of environments.

The interferometric systems as provided herein that may utilize the sensing layer composition provide a high throughput modular design. The systems as provided herein may provide both qualitative and quantitative results from one or more analytes. Particularly, the systems as provided herein may simultaneously provide detection and quantification of one or more analytes from a target sample. According to one embodiment, both qualitative and quantitative results are provided in real-time or near real time.

The interferometric systems as provided herein that may utilize the sensing layer composition can generally include a housing for various detection, analysis and display components. The interferometric system housing includes a rugged, stable, shell or case. The interferometric system housing can withstand hazards of use and cleaning or disinfection procedures of the case surface. The interferometric system housing may be manufactured from a polymer via various techniques such as injection molding or 3D printing. The interferometric system housing may be manufactured to include a coloration that provides the interferometric system housing with a particular color or color scheme.

According to one embodiment, the interferometric systems provided herein include components that are sealed, waterproof or water resistant to the outside environment to minimize opportunities for contamination of a target sample. The overall arrangement of components within the interferometric systems minimize harboring of contamination in any hard-to-reach areas allowing for ease of disinfection.

The interferometric systems provided herein that may utilize the sensing layer composition include a cartridge system. The cartridge systems provided herein integrate with one or more independent or integrated optical waveguide interferometers. The cartridge systems provide efficient sample composition communication through a microfluidic system mounted on or within the cartridge housing. The cartridge is suitable for one or more analytes to be detected in a single sample in a concurrent, simultaneous, sequential or parallel manner. The cartridge systems provided herein may be utilized to analyze in a multiplex manner. That is, one test sample will be tested to determine the presence of multiple analytes at the same time by utilizing a plurality of waveguide channels that interact with the test sample.

The cartridge systems provided herein are easily removable and disposable allowing for overall quick and efficient use without the risk of cross-contamination from a previous target sample. The cartridge may be safely disposed of after a single use. Disposal after a single use may reduce or eliminate user exposure to hazards. According to one embodiment, the cartridge system includes materials that are biodegradable, or recycled materials, to reduce environmental impact. The cartridge system may be cleaned and re-used or otherwise recycled after a single use.

The cartridge system as provided herein may be suited for multiple or one-time use. The single-use cartridge system may be manufactured in a manner such that a buffer solution is pre-loaded in the microfluidic system. By providing the buffer solution pre-loaded in the single-use cartridge system, gas bubbles are reduced or otherwise eliminated. After a single use, the entire cartridge system is safely discarded or recycled for later use after cleaning. Put another way, after introduction and detection of a test sample, the entire single-use cartridge system is not used again and, instead, discarded.

The cartridge systems as provided herein may be suited for multiple uses. According to such an embodiment, the cartridge system may be used one or more times prior to the cartridge system being safely discarded or recycled. The cartridge system may also be cleaned and re-used or otherwise recycled after multiple uses. According to one embodiment, the cartridge system facilitates cleaning and re-tooling to allow the cartridge system to be replenished and returned to operation.

According to one embodiment, the interferometric systems provided herein provide both qualitative and quantitative results at or under 60 minutes after sample introduction to the system. According to one embodiment, both qualitative and quantitative results are provided at or under 30 minutes. According to one embodiment, both qualitative and quantitative results are provided at or under 10 minutes. According to one embodiment, both qualitative and quantitative results are provided at or under 5 minutes. According to one embodiment, both qualitative and quantitative results are provided at or under 2 minutes. According to one embodiment, both qualitative and quantitative results are provided at or under 1 minute.

The interferometric systems as provided herein may be powered via alternating current or direct current. The direct current may be provided by a battery such as, for example, one or more lithium or alkaline batteries. The alternating or direct current may be provided by alternative energy sources such as wind or solar.

According to one embodiment, the interferometric system is stabilized to address vibrational distortions. The system may be stabilized by various means including mechanical, chemical (fluid float or gel pack), computer-assisted system (electronically), or digitally (e.g., via a camera). In some implementations, the systems provided herein allow for point of use assays that are stable in various conditions, including ambient temperature and humidity as well as extreme heat, cold and humidity.

The interferometric systems as provided herein may be equipped with one or more software packages loaded within. The software may be electronically connected to the various system components as provided herein. The software may also be electronically integrated with a display for viewing by a user. The display may be any variety of display types such as, for example, a LED-backlit LCD. The system may further include a video display unit, such as a liquid crystal display (“LCD”), an organic light emitting diode (“OLED”), a flat panel display, a solid-state display, or a cathode ray tube (“CRT”).

According to one embodiment, the interferometric system as provided herein may interface with or otherwise communicate with a transmission component. The transmission component may be in electronic signal communication with both the cartridge system and interferometric system components. The transmission component sends or transmits a signal regarding analyte detection data and quantification data. The transmission of such data may include real-time transmission via any of a number of known communication channels, including packet data networks and in any of a number of forms, including instant message, notifications, emails or texts. Such real-time transmission may be sent to a remote destination via a wireless signal. The wireless signal may travel via access to the Internet via a surrounding Wi-Fi network. The wireless signal may also communicate with a remote destination via Bluetooth or other radio frequency transmission. The remote destination may be a smart phone, pad, computer, cloud device, or server. The server may store any data for further analysis and later retrieval. The server may analyze any incoming data using artificial intelligence learning algorithms or specialized pathological, physical, or quantum mechanical expertise programed into the server and transmit a signal.

According to one embodiment, the transmission component may include a wireless data link to a phone line. Alternatively, a wireless data link to a building Local Area Network may be used. The system may also be linked to Telephone Base Unit (TBU) which is designed to physically connect to a phone jack and to provide 900 MHz wireless communications thereby allowing the system to communicate at any time the phone line is available.

According to one embodiment, the interferometric system may include a location means. Such a location means includes one or more geolocation device that records and transmits information regarding location. The location means may be in communication with a server, either from a GPS sensor included in the system or a GPS software function capable of generating the location of the system in cooperation with a cellular or other communication network in communication with the system. According to a particular embodiment, the location means such as a geolocation device (such as GPS) may be utilized from within its own device or from a mobile phone or similarly collocated device or network to determine the physical location of the cartridge system.

According to one embodiment, the interferometric system contains a geo-location capability that is activated when a sample is analyzed to “geo-stamp” the sample results for archival purposes. According to one embodiment, the interferometric system contains a time and date capability that is activated when a sample is analyzed to time stamp the sample results for archival purposes.

The interferometric systems provided herein may interface with software that can process the signals hitting the detector unit. The cartridge system as provided herein may include a storage means for storing data. The storage means is located on or within the cartridge housing or within the interferometric system housing. The storage means communicates directly with electronic components of the interferometric system. The storage means is readable by the interferometric system. Data may be stored as a visible code or an index number for later retrieval by a centralized database allowing for updates to the data to be delivered after the manufacture of the cartridge system. The storage means may include memory configured to store data provided herein.

The data retained in the storage means may relate to a variety of items useful in the function of the interferometric system. According to a particular embodiment, the data may provide the overall interferometric system or cartridge system status such as whether the cartridge system was previously used or is entirely new or un-used. According to a particular embodiment, the data may provide a cartridge system or interferometric system identification. Such an identification may include any series of letter, numbers, or a combination thereof. Such identification may be readable through a quick response (QR) code. The identification may be alternatively memorialized on a sticker located on the cartridge housing or interferometric system housing. According to one embodiment, the cartridge housing contains a bar code or QR code. According to one embodiment, the cartridge system contains a bar code or QR code for calibration or alignment. According to one embodiment, the cartridge system contains a bar code or QR code for identification of the cartridge or test assay to be performed. According to one embodiment, the cartridge system contains a bar code or QR code for identification of the owner and location of where any data generated should be transmitted. A user may scan such a QR code with the interferometric system's external camera prior to use of the system such that identification and transmission may occur (e.g., automatically or upon user direction).

According to a particular embodiment, the data retained in the storage means may provide the number of uses remaining for a multiple-use cartridge system. According to a particular embodiment, the data may provide calibration data required by interferometric system to process any raw data into interpretable results. According to a particular embodiment, such data may relate to information about the analyte and any special processing instructions that can be utilized by the cartridge system to customize the procedure for the specific combination of receptive surface(s) and analyte(s). The interferometric system as provided herein may include electronic memory to store data via a code or an index number for later retrieval by a centralized database allowing for updates to the data to be delivered after the manufacture of the cartridge system.

The interferometric system may include a memory component such that operating instructions for the interferometric system may be stored. All data may be stored or archived for later retrieval or downloading onto a workstation, pad, smartphone or other device. According to one embodiment, any data obtained from the system provided herein may be submitted wirelessly to a remote server. The interferometric system may include logic stored in local memory to interpret the raw data and findings directly, or the system may communicate over a network with a remotely located server to transfer the raw data or findings and request interpretation by logic located at the server. The interferometric system may be configured to translate information into electrical signals or data in a predetermined format and to transmit the electrical signals or data over a wireless (e.g., Bluetooth) or wired connection within the system or to a separate mobile device. The interferometric system may perform some or all of any data adjustment necessary, for example adjustments to the sensed information based on analyte type or age or may simply pass the data on for transmission to a separate device for display or further processing.

The interferometric systems provided herein may include a processor, such as a central processing unit (“CPU”), a graphics processing unit (“GPU”), or both. Moreover, the system can include a main memory and a static memory that can communicate with each other via a bus. Additionally, the system may include one or more input devices, such as a keyboard, touchpad, tactile button pad, scanner, digital camera or audio input device, and a cursor control device such as a mouse. The system can include a signal generation device, such as a speaker or remote control, and a network interface device.

According to one embodiment, the interferometric system may include color indication means to provide a visible color change to identify a particular analyte. According to one embodiment, the system may include a reference component that provides secondary confirmation that the system is working properly. Such secondary confirmation may include a visual confirmation or analyte reference that is detected and measured by the detector.

The interferometric system as provided herein may also include a transmitting component. The transmitting component may be in electronic signal communication with the detector component. The transmitting component sends or transmits a signal regarding analyte detection and quantification data. The transmission of such data may include real-time transmission via any of a number of known communication channels, including packet data networks and in any of a number of forms, including text messages, email, and so forth. Such real-time transmission may be sent to a remote destination via a wireless signal. The wireless signal may travel via access to the Internet via a surrounding Wi-Fi network. The wireless signal may also communicate with a remote destination via Bluetooth or other radio frequency transmission. The remote destination may be a smart phone, pad, computer, cloud device, or server. The server may store any data for further analysis and later retrieval. The server may analyze any incoming data using artificial intelligence learning algorithms or specialized pathological, physical, or quantum mechanical expertise programed into the server and transmit a signal.

According to one embodiment, the interferometric system includes a wireless data link to a phone line. Alternatively, a wireless data link to a building Local Area Network may be used. The system may also be linked to Telephone Base Unit (TBU) which is designed to physically connect to a phone jack and to provide 900 MHz wireless communications thereby allowing the system to communicate at any time the phone line is available.

According to one embodiment, the system may also include geolocation information in its communications with the server, either from a GPS sensor included in the system or a GPS software function capable of generating the location of the system in cooperation with a cellular or other communication network in communication with the system. According to a particular embodiment, the system may include a geolocation device (such as GPS or RFID) either from within its own device or from a mobile phone or similarly collocated device or network to determine the physical location of the system.

According to one embodiment, the interferometric system includes an external camera. The external camera may be at least partially located within the interferometric system housing but include a lens exposed to the exterior of the housing such that the external camera may take photos and video of a target sample prior to collection (e.g., soil, plant, etc.). The external camera may capture video or images that aid in the identification of an analyte and confirmation of the resulting data. The external camera may also capture video images that aid in selecting a proper remedial measure. The external camera may capture video or images that aid in the identification of a target sample or source thereof.

The external camera may capture video or images in connection with scanning and identifying a QR code (such as a QR code on an external surface of a cartridge housing). When located on an external surface of the cartridge housing, the QR code may also aid in identifying ownership of generated data and transmission of such data to a correct owner.

According to one embodiment, the cartridge system contains a geo-location capability that is activated when a sample is analyzed to “geo-stamp” the sample results for archival purposes. According to one embodiment, the cartridge system contains a time and date capability that is activated when a sample is analyzed to time stamp the sample results for archival purposes. According to one embodiment, the cartridge system includes materials that are biodegradable, or recycled materials, to reduce environmental impact. Any used cartridge system provided herein may be disposed of in any acceptable manner such as via a standard biohazard container. According to one embodiment, the cartridge system facilitates cleaning and re-tooling to allow the cartridge system to be replenished and returned to operation.

According to one embodiment, the cartridge system is stabilized to address vibrational distortions. The system may be stabilized by various stabilization means including mechanical (alignment means as provided herein), chemically (fluid float or gel pack), computer-assisted system (electronically), or digitally (e.g., via a camera or digital processing).

### Microfluidic System Overview—Single-Use Cartridge System

The single-use cartridge system provided herein includes a microfluidic system for communicating or otherwise providing a means for test sample and buffer to mix thereby resulting in a test sample. The microfluidic system causes the test sample to move through the detection region to allow for detection and analysis of one or more analytes. The microfluidic system includes an injection port for introduction of a test sample. The injection port may optionally include a check valve. The microfluidic system further includes a first microchannel section having a first end attached in communication with the injection port check valve and a second end in communication with a mixing bladder. According to one embodiment, the first microchannel section contains a filter to remove materials not capable of detection and quantification. The mixing bladder is sized, shaped and otherwise configured to store buffer. The mixing bladder is sized, shaped and otherwise configured to aid in mixing buffer and test sample to form the test sample. The mixing bladder may be bypassed such that the test sample may be automatically discharged or allowed to proceed through the microfluidic system. The mixing bladder may include a temperature control means in the form of a metal coil wrapped around the mixing bladder such that the temperature control means is heated upon introduction of an electric current.

The microfluidic system further includes second microchannel section having a first end attached in communication with the mixing bladder and a second end attached in communication with a flow cell having at least one detection microchannel. By including multiple two or more detection microchannels, the cartridge system is particularly suited for high throughput and improved testing efficiency by being able to detect and quantify analyte in more than one test sample.

The microfluidic system further includes at least one pump. Suitable pumps include micropumps such as, but are not limited to, diaphragm, piezoelectric, peristaltic, valveless, capillary, chemically-powered, or light-powered micropumps. According to an alternative embodiment, the microfluidic system further includes at least one pump that is a, positive-displacement pump, impulse pump, velocity pump, gravity pump, steam pump, or valve-less pump of any appropriate size. According to a single-use embodiment of the cartridge system, the cartridge system contains at least one pump located within the cartridge housing. According to one embodiment of a single-use cartridge system, the pump overlays or otherwise engages or touches the first microchannel section, second microchannel section and mixing bladder.

The microfluidic system of the single-use cartridge system as provided herein may be manufactured and packaged under negative pressure or vacuum sealed. In this manner, the negative pressure allows for a test sample to be pulled in and become self-loading upon introduction of the test sample. The negative pressure further allows for a test sample to be pulled in in the microfluidic system to reduce, avoid or eliminate bubble formation upon introduction of the test sample. According to an alternative embodiment, the microfluidic system is manufactured and packaged under a positive pressure. According to either embodiment, the microfluidic system of a single-use cartridge system may be pre-loaded with a buffer solution at the time of manufacture. The buffer may be custom designed or designated for a particular analyte detection. Buffer solution that is used (i.e., buffer waste) and resulting test sample composition waste may be contained permanently in the single-use cartridge system, recycled, or otherwise disposed of.

According to one embodiment, the pump can be powered by a battery or electricity transferred from the testing device. Alternatively, the energy to power the pump can be mechanically transferred by direct force, electromagnetic induction, magnetic attraction, audio waves, or piezo electric transfer. According to one embodiment, the cartridge system includes at least one pulse dampening component such as a regulator or accumulator or bladder.

### Microfluidic System Overview—Multiple-Use Cartridge System

The multiple-use cartridge system provided herein includes a microfluidic system for communicating or otherwise providing a means for a test sample to move through the cartridge system and allow for detection and analysis of one or more analytes. According to a particular embodiment, the test sample and test sample are air or liquid. An ingress port is located on a front surface of the multiple-use cartridge system. The ingress port is in communication with a first microchannel section having a first end attached in communication with an ingress port check valve and a second end in communication with second microchannel section. A filter may be located anywhere within the first microchannel section.

The second microchannel section includes a first end in communication with the first microchannel section and a second end in communication with a flow cell having at least one detection microchannel. The cartridge system includes a detection region that accommodates or is otherwise adapted to receive the chip and flow cell wafer.

The detection microchannel is in communication with a first end of a third microchannel section. The third microchannel section includes a flow electrode to approximate flow rate and is correlated with measured impedance. The third microchannel section includes a second end in communication with the first end of a fourth microchannel. The fourth microchannel includes a second end in communication with a check valve which, in turn, is in communication with an egress port. The chip utilized in the multiple-use embodiment may be removable from the cartridge system.

The microfluidic system further includes at least one pump. Suitable pumps include micropumps that include, but are not limited to, diaphragm, piezoelectric, peristaltic, valveless, capillary, chemically-powered, or light-powered micropumps. According to an alternative embodiment, the microfluidic system further includes at least one pump that is a positive-displacement pump, impulse pump, velocity pump, gravity pump, steam pump, or valve-less pump of any appropriate size. According to one multiple-use embodiment of the cartridge system, the cartridge system contains at least one pump located outside (external to) the cartridge housing but in communication with the microfluidic system. The external pump may be utilized to move test sample through the microfluidic system to aid in removal of air or bubble that may be present in a liquid test sample prior to use. According to one embodiment, the cartridge system contains at least one pump dampening device.

The cartridge systems provided herein may utilize the pump to manipulate the communication of test sample throughout the microfluidic system. The pump speed may be adjusted to create optimal conditions such that test sample flow is at a rate high enough to overcome the analyte diffusion barrier layer in conditions that may be mass transport limited while also not being so rapid such that the buffer carries the analyte past the receptors without enough time to meet the kinetic demands that binding requires.

According to one embodiment, the pump causes or otherwise aids movement of test sample through the microchannels as well as the mixing bladder, when present. According to one embodiment, the pump is configured to deliver a test sample flow rate through the interferometric system of from about 0.05 mL/min to about 5.0 mL/min. According to one embodiment, the pump is configured to deliver a test sample flow rate through the interferometric system of up to about 5.0 mL/min. According to one embodiment, the pump is configured to deliver a test sample flow rate through the interferometric system of about 0.25 mL/min to about 2.5 mL/min. According to one embodiment, the pump is configured to deliver a test sample flow rate through the interferometric system of about 0.1 mL/min to about 1.0 mL/min.

### Handheld Interferometric System—Exemplary Embodiment

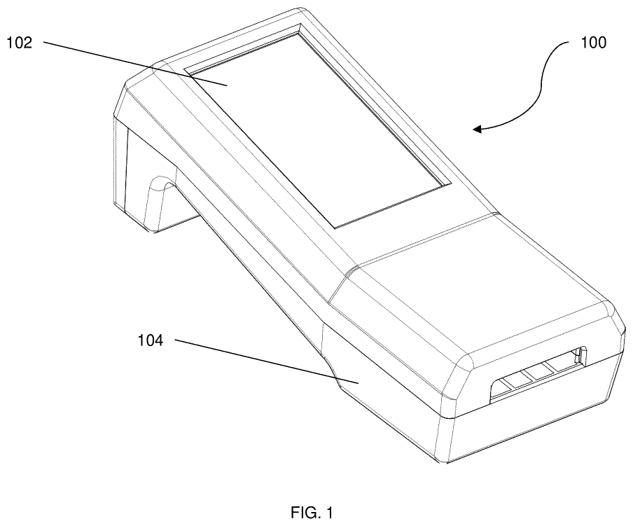

FIG. 1 illustrates a perspective view of one embodiment of a portable interferometric system 100 as provided herein. The portable interferometric system 100 may include a display unit 102. The portable interferometric system 100 may include a housing 104 adapted to fit within a user's hand.

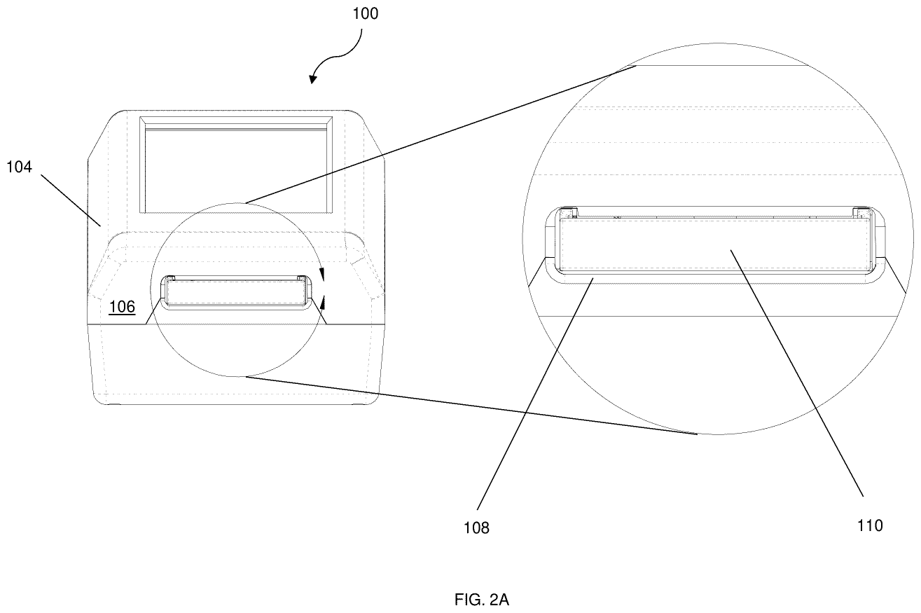

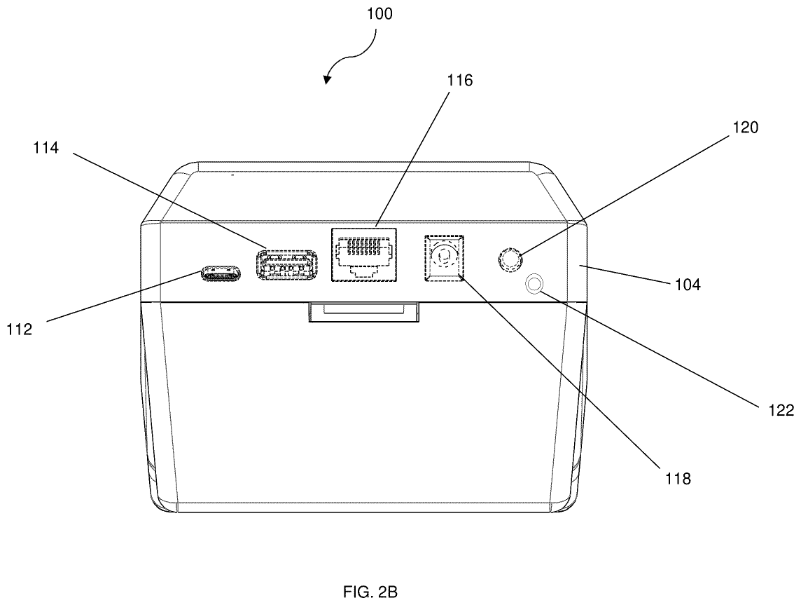

FIG. 2A illustrates a front view of one embodiment of a portable interferometric system 100 that utilizes the cartridge systems provided herein. The housing 104 includes an external front surface 106 defining an opening 108 adapted to receive the cartridge system provided herein. The opening 108 aids in the alignment and proper position of the cartridge system as provided herein within the handheld interferometric system 100. The opening 108 may optionally include a flap 110 that shields or covers the opening 108 when the cartridge is not inserted. The flap 110 may be hinged on any side so as to aid in the movement of the flap 110 from a first, closed position to a second, open position upon insertion of the cartridge system.

FIG. 2B illustrates a rear view of one embodiment of a portable interferometric system 100 as provided herein. The housing 104 is adapted to include USB Type C 112, USB Type A 114, data or phone line inlet 116, power cord inlet 118, power switch 120, and external camera or other light sensitive device 122.

### Chip Overview

As previously noted, the cartridge systems provided herein further includes a detection region. This detection region accommodates or is otherwise adapted to receive an interferometric chip and flow cell wafer. The flow cell wafer includes at least one detection microchannel. The flow cell wafer is located directly above the chip. The detection microchannel may be etched onto a flow cell wafer having a substantially transparent or clear panel or window. The detection microchannel aligns with each waveguide channel in the chip.

In use, a light signal may be emitted from a light unit located in the interferometric system. The light enters flow through entry gradients in the chip and through one or more waveguide channels. According to a particular embodiment, there may be two or more waveguide channels to determine the presence of a separate analyte that each of the individual waveguide channels alone would not have been able to identify alone. The evanescent field is created when the light illuminates the waveguide channel. The light signal is then directed by exit gradients to a detector unit such as a camera unit. The detector unit is configured to receive the light signal and detect an analyte present in a test sample. The chip may further include a reference waveguide channel.

### Sensing Layer Composition

The present disclosure relates to a sensing layer composition that is configured for use in an interferometric system such as the interferometric system described herein. The sensing layer composition is particularly suited for application to at least one surface of one or more waveguide channels on a waveguide chip and is designed to detect target analytes by producing a measurable shift in the optical interference signal. The sensing layer composition includes at least one polymeric receptor and at least one amino silane and is formulated to bind or otherwise be selectively disturbed by one or more analytes of interest. According to one embodiment, the analyte binding alters the local refractive index of the waveguide, producing an optical signal that can be detected by the interferometric system. In some embodiments, the interferometric system is an optical interferometric system.

In one embodiment, the polymeric receptor of the sensing layer composition includes a cyclodextrin derivative that is substituted with one or more carboxylic acid functional groups. The cyclodextrin component is selected to enable hydrophobic inclusion of fluoro-containing compounds and to support ionic interactions through the substituted carboxyl groups. These functional groups also enable covalent coupling of the polymeric receptor to the amino silane. In some embodiments, the cyclodextrin derivative is a cyclodextrin substituted at the primary hydroxyl position with a carboxylic acid moiety, which enhances its ionic interaction capability with acidic analytes such as perfluoroalkyl substances. In some embodiments, the cyclodextrin derivative is α, β or γ-cyclodextrin substituted at the primary hydroxyl position with a carboxylic acid moiety.

The amino silane in the sensing layer composition is provided to facilitate immobilization of the receptor on a solid substrate, such as a silicon dioxide-coated waveguide. A suitable amino silane includes 3-aminopropyltriethoxysilane (APTES), although other aminosilanes may also be used. The amino silane may be selected for its ability to undergo hydrolysis and condensation with hydroxyl groups present on silicon dioxide surfaces, forming a covalent siloxane linkage that serves as an anchoring layer for the sensing layer composition. According to one embodiment, the sensing layer composition may include from about 0.1 percent by weight to about 95 percent by weight of the amino silane, based on the total weight of the sensing layer composition.

According to one embodiment, the polymeric receptor is chemically modified with one or more substituent groups. Exemplary substituent groups include, but are not limited to, succinic acid moieties and fluorinated alkyl chains. The one or more substituent groups enhance aqueous solubility, improve surface orientation, and promote optimal exposure of the cyclodextrin cavity toward incoming target analytes. According to one embodiment, the one or more substituent groups further improve the chemical compatibility between the cyclodextrin cavity and fluoro-containing substances by increasing local fluoro-containing substance concentration near the receptor site, thereby enhancing binding efficiency and sensitivity.

In some embodiments, the succinic acid moiety may be introduced to increase the number of carboxylic acid groups on the cyclodextrin derivative, thereby enhancing opportunities for covalent coupling, hydrogen bonding, or ionic interaction with target analytes. Suitable succinic acid moieties include, but are not limited to, succinic acid itself, succinic anhydride, maleic acid, malonic acid, and glutaric acid. These moieties may be coupled to the cyclodextrin via esterification, amidation, or anhydride ring-opening reactions with primary hydroxyl groups on the cyclodextrin molecule. According to one embodiment, succinic anhydride may be reacted with a cyclodextrin to yield a mono- or poly-substituted cyclodextrin derivative bearing pendant carboxylic acid groups, thereby enhancing the hydrophilicity and conjugation potential of the receptor toward target analytes. According to one embodiment, succinic anhydride may be reacted with α, β or γ-cyclodextrin to yield a mono- or poly-substituted cyclodextrin derivative. According to one embodiment, the polymeric receptor is 3-aminopropyltriethoxysilane (APTES) and the amino silane is a carboxylic acid-substituted cyclodextrin derivative such as succinyl β-cyclodextrin.

In other embodiments, fluorinated alkyl chains may be attached to the cyclodextrin derivate to introduce fluorophilic interactions that facilitate selective binding of fluoro-containing analytes, including perfluoroalkyl and polyfluoroalkyl substances (PFAS). Suitable fluorinated alkyl moieties include perfluorobutyl groups, perfluorooctyl groups, and longer perfluorinated chains. Representative reagents for introducing fluorinated groups include perfluorobutyl alcohol, perfluorooctyl iodide, 1H,1H,2H,2H-perfluorodecanol, and perfluorinated thiols such as perfluorohexanethiol or perfluorodecanethiol. These reagents may be coupled to the cyclodextrin scaffold via ester, ether, amide, thiol, or urethane linkages depending on the reactive functionality available on the fluorinated species and the derivatized cyclodextrin.

According to one embodiment, fluorinated alcohols such as perfluorooctanol or perfluorodecanol may be esterified with carboxyl-substituted cyclodextrins using carbodiimide coupling agents such as 1-ethyl-3-(3-dimethylaminopropyl) carbodiimide (EDC) in the presence of N-hydroxysuccinimide (NHS). Alternatively, perfluoroalkyl isocyanates may be reacted with hydroxyl groups on the cyclodextrin to form stable urethane bonds. In embodiments utilizing thiol-reactive species, the fluorinated moiety may contain a terminal thiol group that reacts with maleimide-functionalized or acrylate-modified cyclodextrins to form thioether linkages via thiol-Michael addition or thiol-ene reactions.

According to one embodiment, the use of fluorinated alkyl chains enhances hydrophobic interaction between the sensing layer composition and target analytes (e.g., PFAS), while the cyclodextrin cavity provides a complementary host-guest interaction. By combining hydrophobic fluorophilic domains with carboxylic acid functionality, the modified cyclodextrin derivatives may offer dual-mode binding and binding stabilization through hydrophobic, electrostatic, size inclusion, fluorophilic, ionic fluorophilic, ionic interactions, van der Waals interactions, or any combination thereof.

According to one embodiment, both succinic acid moieties and fluorinated alkyl chains may be introduced on the same cyclodextrin derivative to enhanced analyte selectivity and film adhesion characteristics. Such multifunctionalized cyclodextrin derivatives may be employed to optimize sensing performance, regeneration time, and stability.

The polymeric receptor of the sensing layer composition may be covalently linked to the amino silane via a carbodiimide-based coupling reaction. In one embodiment, the cyclodextrin derivative may be first activated using 1-ethyl-3-(3-dimethylaminopropyl) carbodiimide (EDC) and N-hydroxysuccinimide (NHS) to form a reactive ester intermediate, which is then reacted with the amine group of the amino silane to form a stable amide bond. The use of excess EDC and NHS ensures efficient activation of the carboxyl groups and promotes uniform coupling. The resulting receptor-functionalized film forms part of the sensing layer composition and exhibits strong adhesion to the waveguide channel surface. The sensing layer composition is chemically suited to bind fluoro-containing analytes through a combination of hydrophobic and ionic interactions.

The concentration of the polymeric receptor within the sensing layer composition may vary depending on the desired binding capacity and optical sensitivity. In one embodiment, the sensing layer composition comprises from about 0.1 percent by weight to about 95 percent by weight of the polymeric receptor, based on the total weight of the sensing layer composition. More typically, the sensing layer composition comprises between 0.1 percent by weight and 50 percent by weight of each of the amino silane and the polymeric receptor.

The sensing layer composition may be designed to exhibit hydrophilic behavior and may be compatible with aqueous or aqueous-organic buffer systems. In one embodiment, the sensing layer composition is applied in combination with a buffer solution containing no more than 5 percent by volume of an organic solvent, such as ethanol or methanol. The sensing layer composition is particularly suited for binding fluoro-containing analytes in water-based samples, including environmental or biological fluids. The sensing layer composition enables selective detection of perfluoroalkyl and polyfluoroalkyl substances (PFAS), including but not limited to perfluorooctanoic acid (PFOA), perfluorooctane sulfonic acid (PFOS), perfluorononanoic acid (PFNA), hexafluoropropylene oxide dimer acid (HFPO-DA, also known as GenX), perfluorohexane sulfonic acid (PFHxS), perfluorobutanoic acid (PFBA), perfluorodecanoic acid (PFDA), and perfluorobutane sulfonic acid (PFBS).

Upon binding of one or more of the aforementioned analytes, the sensing layer composition induces a detectable change in the local refractive index of the waveguide surface. This change causes a shift in the interference pattern produced by the optical interferometric system, which can be correlated with analyte presence and concentration. The sensing layer composition may be deposited on the waveguide chip using various coating methods, including spin coating, dip coating, spray deposition, or microfluidic delivery. According to one embodiment, the sensing layer composition forms a continuous film that is uniform, optically stable, and mechanically robust.

The sensing layer composition supports reversible binding to the target analytes, allowing the sensor surface to be regenerated between uses. Regeneration may be achieved by rinsing with buffer solution and re-equilibration, with surface recovery occurring in less than 30 minutes in some embodiments. The sensing layer composition also exhibits long-term stability under continuous measurement conditions, maintaining its optical and chemical properties for a period of at least four hours in buffered aqueous environments.

In one embodiment, the sensing layer composition is applied to both sensing and reference channels of the interferometric waveguide chip, allowing the system to operate in a differential mode. This configuration improves measurement accuracy by enabling real-time correction of environmental drift and nonspecific effects. The sensing layer composition provides a dynamic response range suitable for detecting PFAS concentrations between approximately five parts per billion and five hundred parts per billion. The response of the sensing layer composition under these conditions is reproducible, with a coefficient of variation of less than ten percent in repeated measurements.

Alternative embodiments of the sensing layer composition may employ cyclodextrin derivatives bearing other functional groups, such as sulfonate, phosphate, or hydroxyalkyl substituents. Alternative silane coupling agents, crosslinking chemistries, or waveguide materials may also be used depending on the requirements of the sensing platform. The sensing layer composition described herein is broadly applicable to optical sensing of environmental contaminants and is well suited for integration into portable or field-deployable interferometric sensor systems.

According to one embodiment, the sensing layer composition is configured to bind or otherwise be selectively disturbed by one or more analytes in a test sample at a flow rate through the interferometric system of from about 0.05 mL/min to about 5.0 mL/min. According to one embodiment, the sensing layer composition is configured to bind or otherwise be selectively disturbed by one or more analytes in a test sample at a flow rate through the interferometric system of up to about 5.0 mL/min. According to one embodiment, the sensing layer composition is configured to bind or otherwise be selectively disturbed by one or more analytes in a test sample at a flow rate through the interferometric system of from about 0.25 mL/min to about 2.5 mL/min. According to one embodiment, the sensing layer composition is configured to bind or otherwise be selectively disturbed by one or more analytes in a test sample at a flow rate through the interferometric system of from about 0.1 mL/min to about 1.0 mL/min.

### Flow Cell Overview

Each of the cartridge systems described herein include a flow cell having at least one detection microchannel adapted to communicate with one or more test samples flowing through a waveguide channel in a chip beneath the flow cell. According to one embodiment, the cartridge systems may include at least two, at least three, or at least four detection microchannels with each detection microchannel adapted to communicate one or more test samples allowing detection of the same or different analytes.

Each detection microchannel is located on or within a flow cell manufactured from a wafer. The at least one detection microchannel may be etched, molded or otherwise engraved into one side of the flow cell wafer. Thus, the at least one detection microchannel may be shaped as a concave path as a result of the etching or molding within the flow cell wafer.

The flow cell wafer is oriented above the chip during use such that the detection microchannel may be orientated or otherwise laid out in variety of flow patterns above the waveguide channels. The detection microchannel may be laid out, for example, in a simple half loop flow pattern, serial flow pattern, or in a serpentine flow pattern. The serpentine flow pattern is particularly suited for embodiments where there are multiple waveguide channels that are arranged in a parallel arrangement. By utilizing the serpentine flow pattern, the test composition flows consistently over the waveguide channels without varying flow dynamics.

### Chip, Flow Cell and Optical Assembly—Exemplary Embodiment

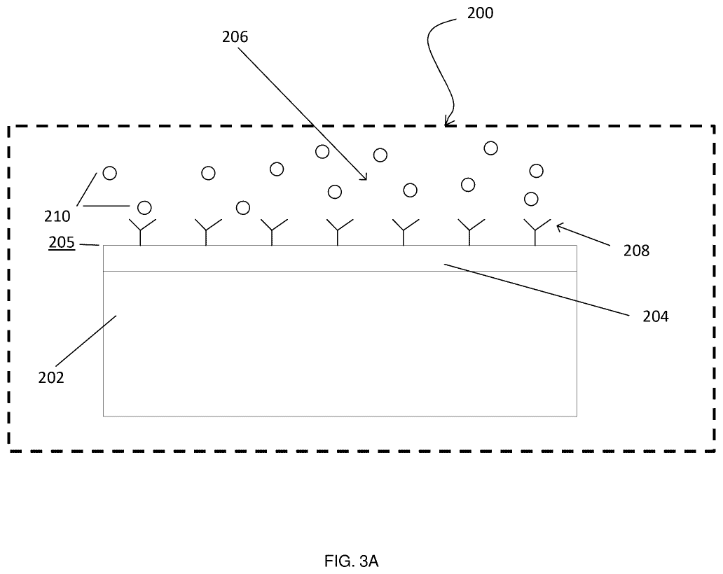

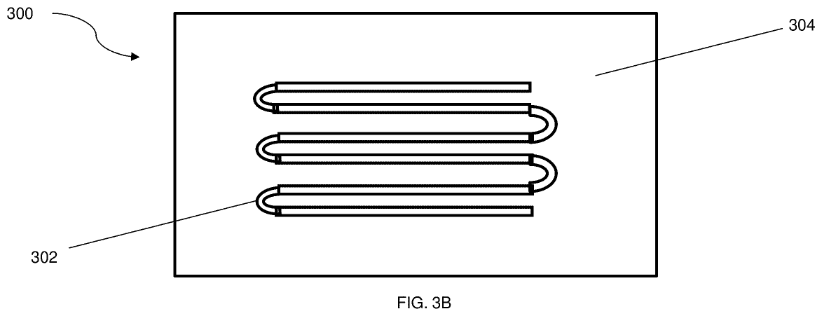

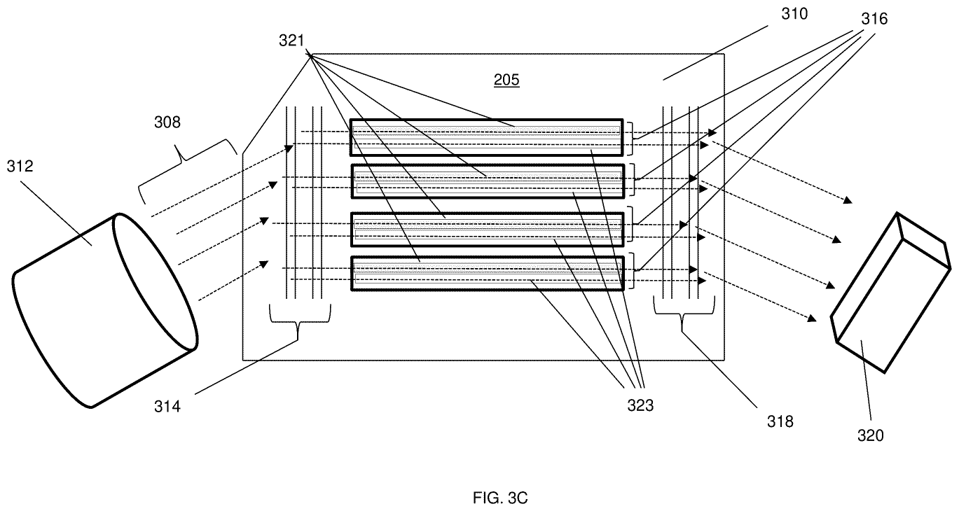

FIG. 3A illustrates a cross-sectional view of an optical detection region 200 of a cartridge system. A chip (or substrate) 202 includes a waveguide channel 204 with a surface 205 (such as the illustrated top surface) of the chip 202. An evanescent field 206 is located above the waveguide channel 204. A sensing layer 208 is adhered to a top side of the waveguide channel 204. As illustrated, analytes 210 are shown that may bind or otherwise immobilized to the sensing layer 208, however, the sensing layer 208 may be adapted to bind any variety of analytes. As such, adjusting or otherwise modifying the sensing layer 208 allows for the cartridge system to be utilized for multiple different types of analytes without having to modify the cartridge system or surrounding interferometric system components. In general use, a light signal (e.g., laser beam) illuminates the waveguide channel 204 creating the evanescent field 206 that encompasses the sensing layer 208. Binding of an analyte impacts the effective index of refraction of the waveguide channel 204.

A bottom view of an exemplary flow cell 300 is illustrated in FIG. 3B. At least one detection microchannel 302 is located on or within a flow cell 300 manufactured from a transparent wafer. The at least one detection microchannel 302 may be etched, molded or otherwise engraved into one side of the flow cell wafer 304. Thus, the at least one detection microchannel 302 may be shaped as a concave path as a result of the etching or molding within the flow cell wafer 304. The flow cell wafer 304 may be manufactured a material such as opaque plastic, or other suitable material. The flow cell wafer 304 may optionally be coated with an anti-reflection composition.

The movement of a light signal 308 (series of arrows) through an optical waveguide chip 310 is illustrated in FIG. 3C. The light signal 308 moves from a light unit 312, such as a laser unit, through a plurality of entry gradients 314 and through one or more waveguide channels 316. Each channel includes a pair of waveguides (321, 323). One of the pair of waveguides 321 is coated with a sensing layer 208 (as indicated by shading in FIG. 3C). The other one of the pair of waveguides 323 is not coated with the sensing layer 208 (serving as a reference). The combination of the light from each in the pair of waveguides (321, 323) create an interference pattern which is illuminated on detector unit 320.

According to a particular embodiment, the two or more waveguide channels 316 are utilized that are able to determine the presence of an analyte that each of the individual waveguide channels 316 alone would not have been able to identify alone. The light signal 308 is then directed by exit gradients 318 to a detector unit 320 such as a camera unit. The detector unit 320 is configured to receive the light signal 308 and detect any analyte present in a target sample composition flowing through the detection microchannel 302 (see FIG. 3B).

The chip 310 includes a combination of substrate 202 (see FIG. 3A), waveguide channel (see FIG. 3A part 204 and FIG. 3C part 316) and sensitive layer 208 (see FIG. 3A). The flow cell 300 (see FIG. 3B) is oriented above the top surface 205 of the chip 310 during use such that the detection microchannel 302 (see FIG. 3B) may be orientated or otherwise laid out in variety of flow patterns above the waveguide channels 316. The detection microchannel 302 may be laid out, for example, in a simple half loop flow pattern, serial flow pattern, or in a serpentine flow pattern as illustrated in FIG. 3B. The serpentine flow pattern is particularly suited for embodiments where there are multiple waveguide channels 316 that are arranged in a parallel arrangement (see FIG. 3C). By utilizing the serpentine flow pattern, the test composition flows consistently over the waveguide channels 316 without varying flow dynamics.

The light signal passes through each waveguide channel as illustrated in FIG. 3C and may combine thereby forming diffraction patterns on the detector unit. The interaction of the analyte 210 (see FIG. 3A) and the sensing layer 208 changes the index of refraction of light in the waveguide channel per Equation 1. The diffraction pattern is moved which is detected by the detector unit. The detector unit as provided herein may be in electronic communication with video processing software. Any diffraction pattern movement may be reported in radians of shift. The processing software may record this shift as a positive result. The rate of change in radians that happens as testing is conducted may be proportional to the concentration of the analyte.

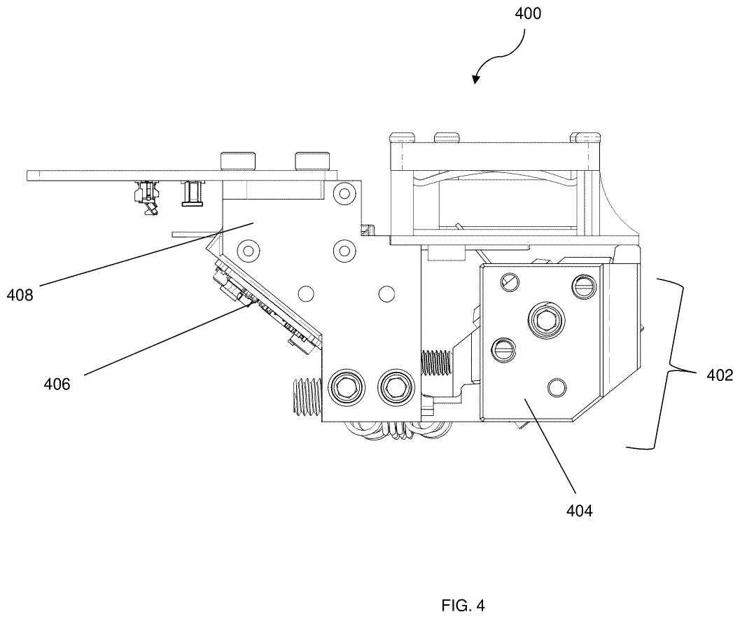

FIG. 4 illustrates a side view of an exemplary embodiment of an optical assembly unit 400 that can be found in the handheld interferometric systems described herein (such as in FIGS. 1-2). The optical assembly unit 400 includes a light unit 402 aligned in a light unit housing 404. The optical assembly unit 400 includes a detector unit 406, such as a camera unit, aligned in a camera unit housing 408.

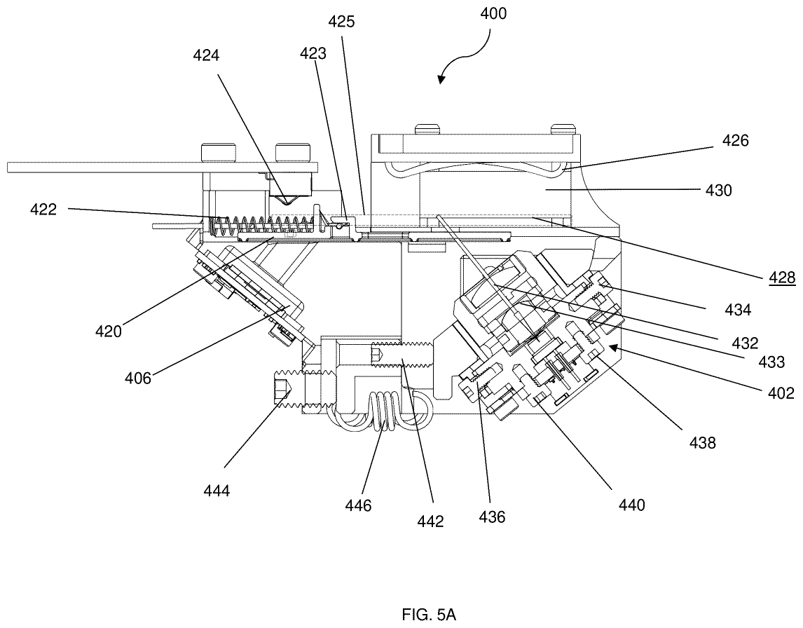

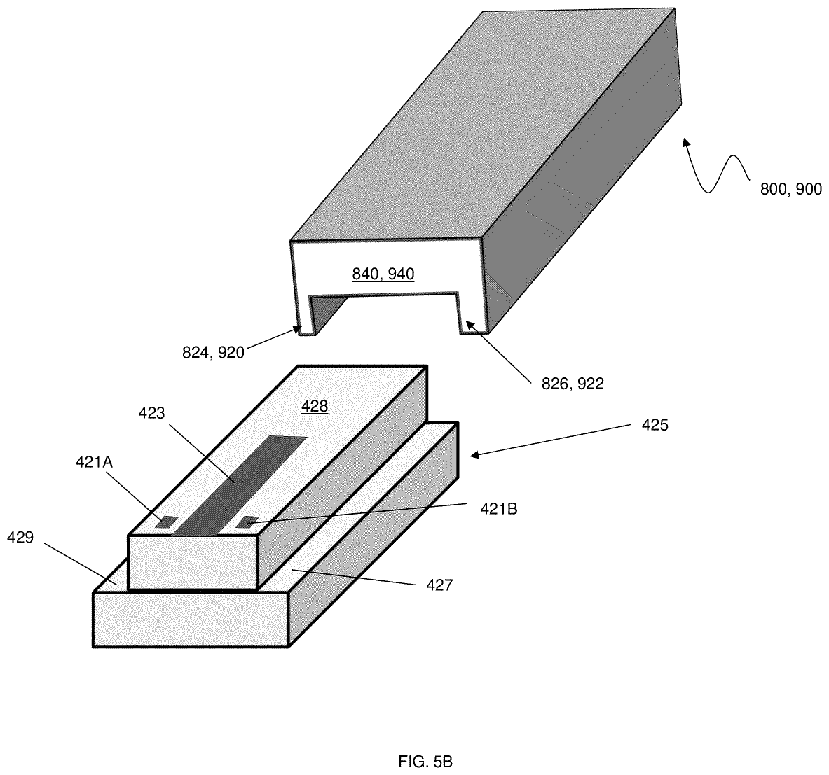

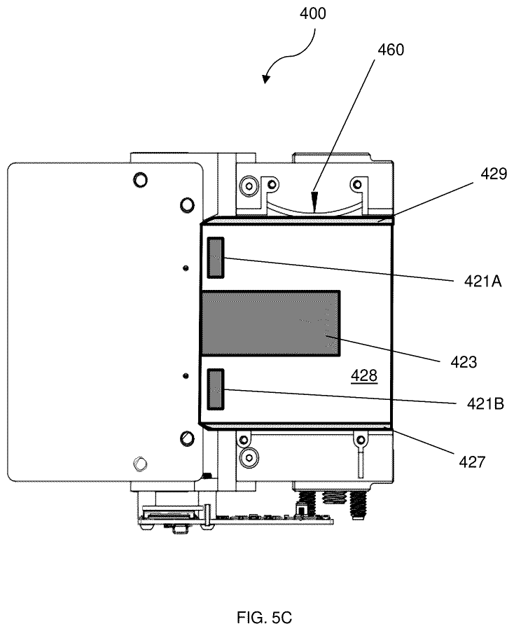

FIG. 5A illustrates a cross-sectional view of the optical assembly unit 400 of FIG. 4. The light unit 402 is situated at an angle relative to the shutter flap element 420. The shutter flap element 420 is adapted to slide open and shut under tension from a shutter spring 422. The shutter flap element 420 is illustrated in a first, closed position with no cartridge system inserted. The shutter flap element 420 includes an upper control arm 423 that is located within a rail portion 425.

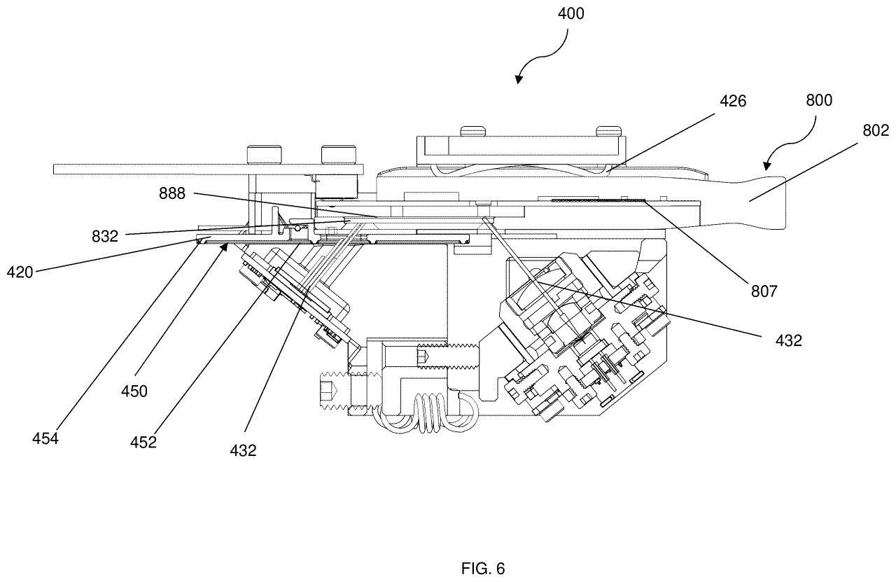

A complimentary communication means 424 extends downward so as to make electronic contact with electronic communications means located on the cartridge housing (see FIGS. 6, 8A and 9A). The complimentary communication means 424 may be metal contacts such that, upon insertion, the metal contacts on the exterior surface of the cartridge housing touch and establish electronic communication between the cartridge system and the remaining components of the interferometric system (e.g., light unit, camera unit, etc.). The complimentary communication means 424, as illustrated, include one or more substantially pointed or “V” shaped so as to push down into or otherwise contact the cartridge housing metal contacts. The number of complimentary communication means 424 may match and align with the number of metal contacts on the exterior surface of the cartridge housing.

At least one downward cantilever bias spring 426 may be located within the optical assembly unit 400 such that, upon insertion of the cartridge through the interferometric system housing opening, the downward cantilever bias spring 426 pushes against a top side of the cartridge housing thereby forcing the cartridge housing against an opposite side or bottom portion or surface 428 of the cartridge recess 430 resulting in proper alignment along a vertical plane (see FIGS. 5A, 5B, 5C and 6).

The light unit 402 is optionally adjustable along various planes for optimal light signal 432 emission. As illustrated, the signal 432 is shown to be emitted and focused by at least one lens 433. A camera unit 406 is situated at an angle relative to the shutter flap element 420 so as to receive the light signal 432 upon exit from the cartridge (see FIG. 6).

A first roll adjustment screw 434 and second roll adjustment screw 436 are located on opposing sides of the light unit 402 for adjusting roll of the light unit 402. A first upward adjustment screw 438 and second upward adjustment screw 440 are located in a parallel manner on each side the light unit 402 for adjusting the light unit 402 towards the cartridge system (i.e., substantially upward). An angle of incidence screw 442 is located against the light unit 402 to allow for adjustments to the angle of incidence for proper coupling angle. A translation screw 444 is located direct communication with the light unit 402 to adjust translation in the X axis. A spring element 446 maintains the position of the light unit 402 against the light unite 402 by assisting the adjustment screws (434, 440), incidence screw 442 and translation screw 444.

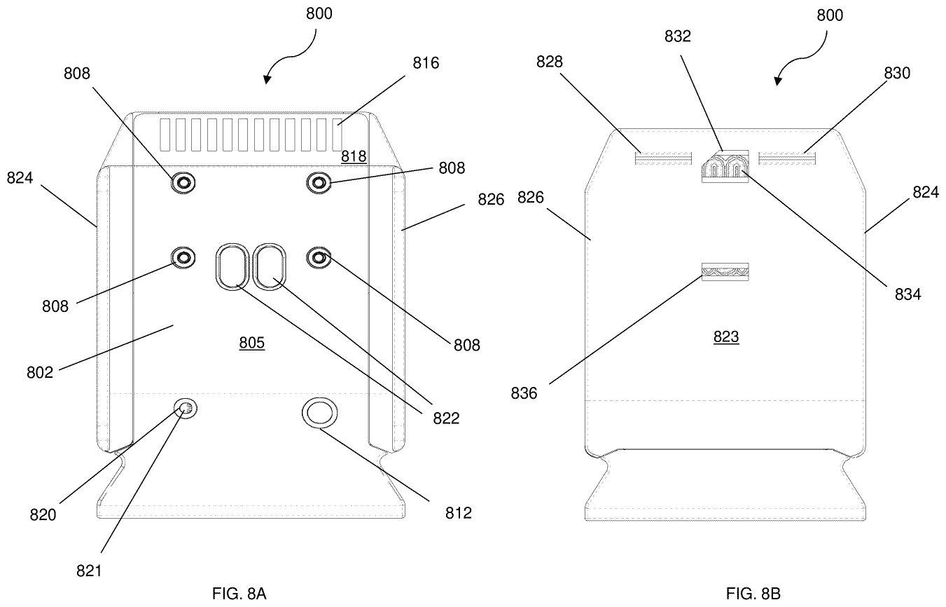

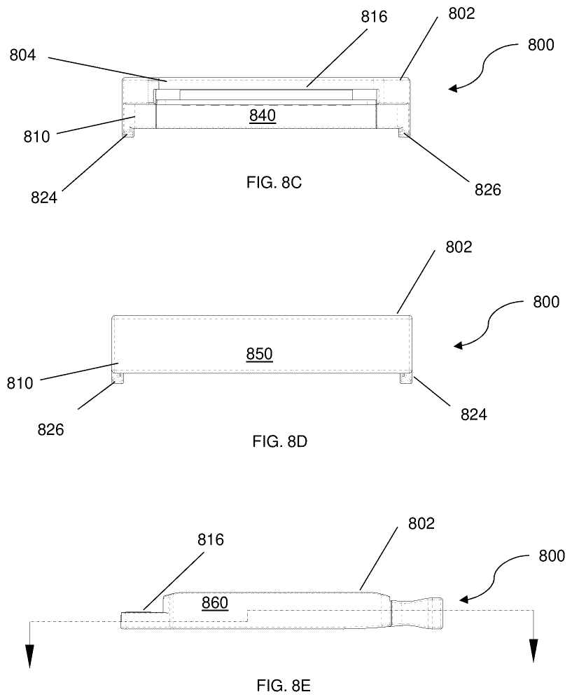

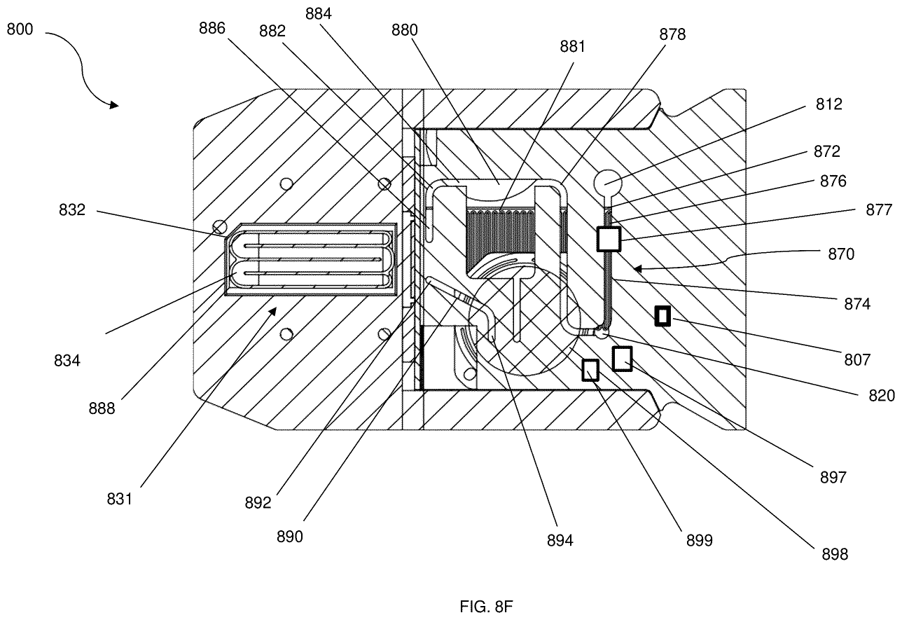

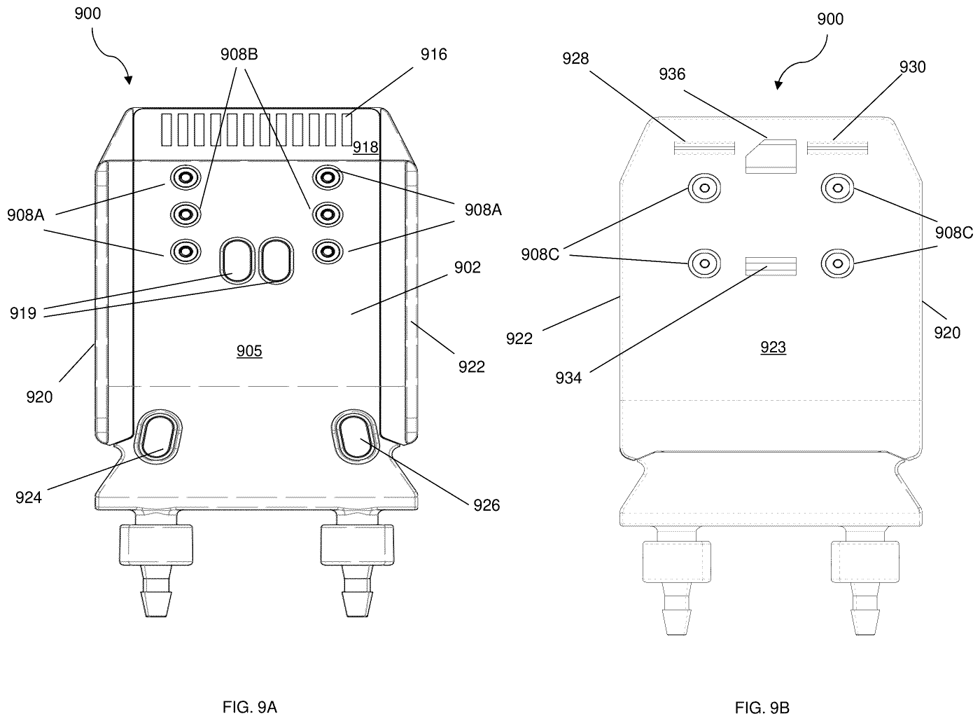

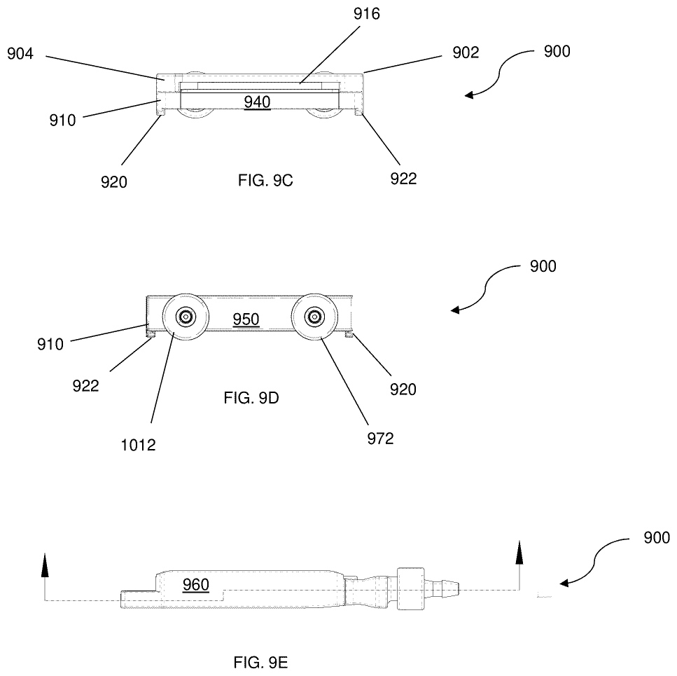

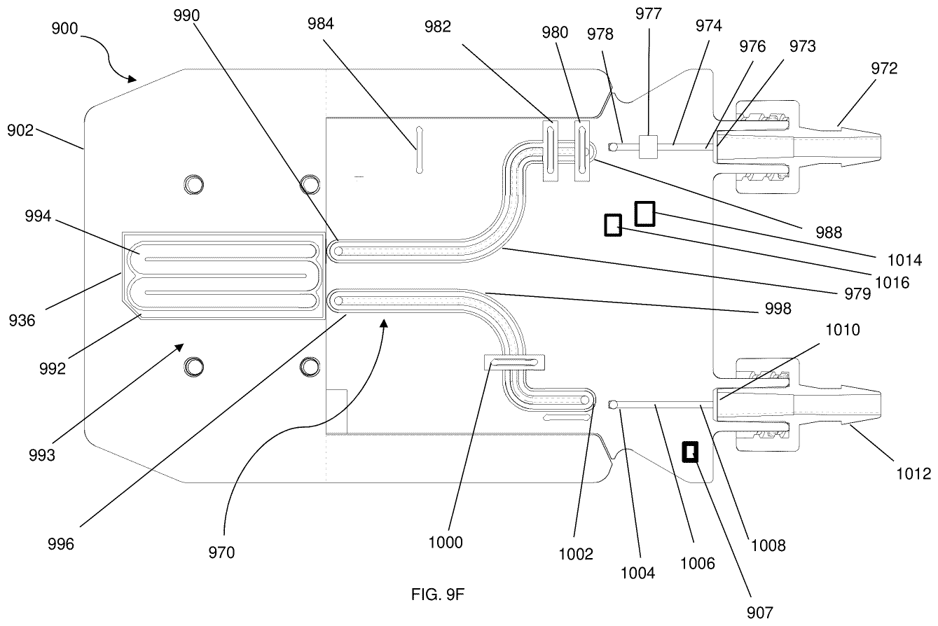

With specific regard to FIGS. 5A, 5B, and 5C, the bottom portion or surface 428 of the cartridge recess 430 further includes alignment means that includes at least one rail portion 425 for engaging both male key portions on the cartridge housing (see 824, 826 of FIG. 8A; see 920, 922 of FIG. 9A). The bottom portion or surface 428 of the cartridge recess 430 includes a first raised surface 421A and second raised surface 421B. A shutter upper control arm 423 is located within the rail portion 425. The rail portion 425 includes a first rail wing 427 and second rail wing 429 adapted to receive and engage the male key portions (see 824, 826 of FIG. 8A; see 920, 922 of FIG. 9A). By including such alignment means, the cartridge systems provided here may only engage in a certain manner thereby preventing incorrect insertion and provided proper optical and microfluidic alignment.

FIG. 6 illustrates a cross-sectional view of the optical assembly 400 of FIG. 5A with one embodiment of a cartridge system 800 inserted in the optical assembly 400. As illustrated, the shutter flap element 420 is pushed backwards upon insertion of the cartridge system 800. While not shown, the shutter spring 422 is compressed backwards. The shutter flap element 420 moves along a track system 450 having a stationary male rail 452 on which a female rail portion 454 slides from a first, closed position with no cartridge system 800 inserted to a second, open position as illustrated in FIG. 6 upon cartridge system 800 insertion.

FIG. 6 further illustrates positioning of the cartridge system 800 in the optical assembly 400. The cartridge system 800 includes an interferometric chip 832 positioned below the flow cell wafer 888. The cartridge system 800 includes storage means 807 as provided herein positioned within the cartridge housing 802. While the cartridge system 800 is illustrated as a single-use system, the alignment and positioning of the single-use cartridge assembly may also apply to the multiple-use cartridge systems provided herein (e.g., see FIGS. 9A-9F).

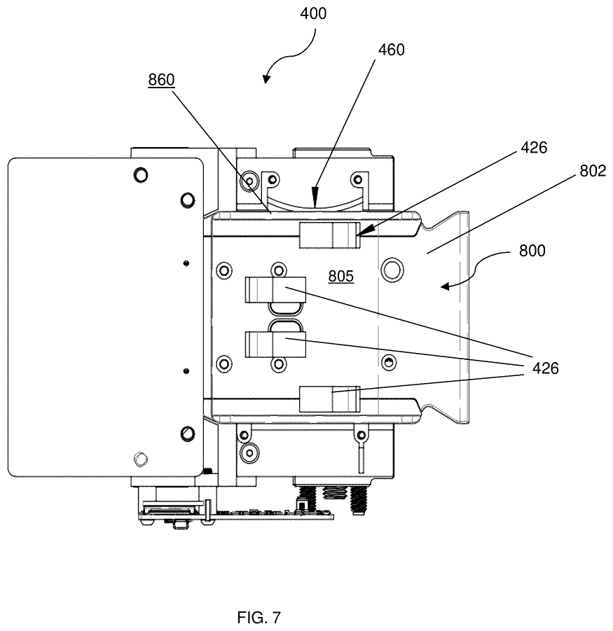

FIG. 7 illustrates a top view of the optical assembly unit 400 of FIG. 5A with one embodiment of a cartridge system 800 inserted in the optical assembly unit 400. The cartridge system 800, as illustrated, is a single-use system, however, a multiple-use system may be inserted in the same manner within the interferometric system. The cartridge system 800 includes a cartridge housing 802 having a top surface 805. The optical assembly unit 400, as illustrated, includes a plurality of cantilever bias springs 426. The optical assembly unit 400 further includes at least one side bias spring 460 such that, upon insertion of the cartridge system 800, the side bias spring 460 pushes against one horizontal side 860 of the cartridge housing thereby forcing the cartridge housing 802 into proper alignment along a horizontal plane.

### Cartridge System Overview

The cartridge systems provided herein includes a cartridge housing. The cartridge housing may be manufactured from any polymer suitable for single or multiple-use. The cartridge may be manufactured according to a variety of additive processing techniques such as 3-D printing. The cartridge may be manufactured via traditional techniques such as injection molding. The polymer may include a coefficient of expansion such that the housing does not expand or contract in a manner that would disrupt alignment of any microfluidic or detection components described herein when the cartridge is exposed to heat or cold environmental conditions.

The cartridge housing may include a light prevention means to aid in reducing, preventing or eliminating ambient, outside light from interfering with the detection of one or more analytes. The light prevention means may include colored cartridge housing (e.g., black colored) that is color dyed or coated during manufacture. According to one embodiment, a dye may be introduced to the polymer to provide a specific color to a region of or the entire cartridge housing. Suitable colors include any color that aids in reducing, preventing or eliminating ambient outside light from interfering with the detection of one or more analytes.

The cartridge systems provided herein further includes a detection region. This detection region accommodates or is otherwise adapted to receive an interferometric chip and flow cell wafer. The flow cell wafer includes at least one detection microchannel. The flow cell wafer is located directly above the chip. The detection microchannel may be etched onto a flow cell wafer having a substantially transparent or clear panel or window. The flow cell wafer, the chip or both the flow cell and chip may be coated with a substance that reduces or eliminates fogging or condensation. According to one embodiment, the chip may be heated to reduce or elimination fogging or condensation.

The cartridge systems provided herein are configured or otherwise adapted or designed to easily insert and instantly align within an interferometric system such as, for example, a hand-held interferometric system. By being configured to allow for instant alignment, no further adjustment is required by a user to align any microfluidic components and any internal detection-related components such as the laser, chip with waveguides and exposed channels in a detection region of the cartridge, optical detector and any other focus-related components in the interferometric system.

The cartridge housing includes dimensions that are complimentary in size and shape to the size and shape to an internal surface defining a recess within an interferometric system. As provided and illustrated in the non-limiting examples herein, the cartridge housing may be generally rectangular in overall shape.

According to one embodiment, the cartridge system may be inserted and removed automatically. According to one embodiment, the cartridge housing contains a bar code or QR code. According to one embodiment, the cartridge system contains a bar code or QR code for calibration or alignment.

To aid in alignment, the cartridge housing includes an alignment means on an external surface of the cartridge housing. The alignment means many take a variety of forms that assure instant alignment of any microfluidic components and any internal detection-related components upon insertion of the cartridge within the interferometric system. The alignment means also aids in the prevention of incorrect orientation assertion within the interferometric system and allows for insertion only after proper alignment is attained. The alignment means further allows for the cartridge system to be stabilized to address vibrational distortions.

The alignment means may include at least one male key portion for engaging and securing within a corresponding female rail located in the interferometric system. The male key portion may be disposed on the bottom surface of the cartridge housing, however, the male key portion may be located on any exterior surface of the cartridge housing. Other suitable alignment means include one or more microswitches or sensing devices that guide the cartridge housing to assure proper alignment.

According to a particular embodiment, the cartridge housing includes a top portion and a bottom portion based on the orientation of insertion in an interferometric system. The top portion may include a top surface defining at least one through hole on at least one external surface of either the top portion or bottom portion. The at least one through hole is adapted to receive a removable fastening means for securing the top portion and bottom portion together. Suitable fastening means include screws or other suitable fastener that may be removed. By allowing the top portion and bottom portion of the cartridge housing to be separated and re-attached, a user may open the cartridge housing to allow for cleaning as well as replacement of the chip.

The cartridge system as provided herein may include a temperature control means to control temperature as well as humidity. The cartridge system as provided herein may include a temperature control means to control test sample temperature. By controlling temperature and humidity around the cartridge system, the interferometric system can provide more repeatable, precise results. According to one embodiment, the cartridge system contains heating capability to facilitate consistent measurement and operation in cold temperatures. By controlling temperature and humidity around the cartridge system, fogging or condensation that causes interference in the detection region of the cartridge system is reduced or otherwise eliminated. The temperature control means may be located on or within the cartridge housing. According to a single-use cartridge system embodiment, the temperature control means is located on or around the mixing bladder of the microfluidic fluid system described herein. The temperature control means may be located on an exterior surface of the cartridge housing. One suitable temperature control means includes a metal coil that is heated upon introduction of an electric current. Another suitable temperature control means includes one or more warming bands or Peltier devices that can provide heating or cooling.

Each of the cartridge systems described herein include a flow cell having at least one detection microchannel adapted to communicate with one or more test samples flowing through a waveguide channel in a chip beneath the flow cell. According to one embodiment, the cartridge systems may include at least two detection microchannels with each detection microchannel adapted to communicate one or more test samples allowing detection of the same or different analytes. According to one embodiment, cartridge system includes a flow cell having at least three detection microchannels with each detection microchannel adapted to communicate one or more test samples allowing detection of the same or different analytes. According to one embodiment, cartridge system includes a flow cell having at least four detection microchannels with each detection microchannel adapted to communicate one or more test samples allowing detection of the same or different analytes.

### Cartridge System—Exemplary Embodiments

An exemplary embodiment of a single-use cartridge system 800 is illustrated in FIGS. 8A-F. A top view of a cartridge system 800 is provided in FIG. 8A. The cartridge system 800 includes a cartridge housing 802 as described herein. The housing 802 includes a top portion 804 (see FIG. 8C) having a top surface 805. The top surface 805 includes four heat stake posts 808 for joining the top portion 804 of the cartridge housing 802 to a bottom portion 810 (See FIG. 8C) of the cartridge housing 802. By utilizing heat stake posts 808, the top portion 804 may be permanently joined to a bottom portion 810 of the cartridge housing 802. The top surface 805 includes an injection port 812 for introduction of a test sample.

The cartridge housing 802 further includes an electronic communication means 816 located on a second external surface 818 that is on a different horizontal plane from the top surface 805. The electronic communication means 816 as illustrated includes a plurality of metal contacts.

The cartridge system further includes a vent port 820. The vent port 820 allows for any air in the microfluidic system 870 (see FIG. 8F), such as in the form of bubbles, to exit. The vent port 820 may include a vent cover 821 over the vent port 820. The vent cover 821 may be fabricated from a material that repels liquid while allowing air or vapor to pass through such as, for example, expanded polytetrafluoroethylene (commercially available as Goretex®. The vent cover 821 allows for air purging from the cartridge system 800 but will not allow fluid to pass through such as when a vacuum is applied to prime the microfluidic system 870. In this way, bubble formation in a liquid test sample is removed or otherwise avoided. The top surface 805 also includes two port seals 822. The port seals 822 may be made from rubber and provides sealing of the microfluidic system 870 within the cartridge system 800.

FIG. 8B illustrates a view of the bottom surface 823 of one embodiment of a single-use cartridge system 800. The bottom surface 823 includes a first male key portion 824 and a second male key portion 826. The male keying portions (824, 826) engage with a corresponding rail portion (425—See FIGS. 5A and 5B) located in the cartridge recess 430 of the optical assembly 400. The bottom surface 823 further defines a first detent 828 and a second detent 830. The detents (828, 830) engage with or otherwise receive a corresponding first raised surface and a second raised surface (421A, 421B) inside the cartridge recess 430 of the optical assembly 400 (see FIGS. 5A, 5B and 5C). When engaged with the first detent 828 and second detent 830, the first raised surface and second raised surface (421A, 421B) aid in securing the cartridge system 800 within the cartridge recess 430.

The chip 832 is substantially transparent and allows the light signal to enter, interact with one or more waveguides channels (See FIG. 3C) and allow for binding of analyte flowing within the at least one detection microchannel 834 within the flow cell wafer 888 (See FIG. 8F).

The bottom surface 823 further defines a light inlet slot 836. The light inlet slot 836 allows for a light signal to enter the cartridge system 800. Particularly, the light inlet slot 836 allows for an light signal to enter the chip 832 and for the light signal to move through any waveguide channels (not shown; see e.g., part 316 of FIG. 3C) in the chip 832 while interacting with analytes in the at least one detection microchannel 834 before the light signal is deflected by one or more gratings (not shown) down to the detector unit 406 (FIG. 5A; see also (320) of FIG. 3C).

FIG. 8C illustrates a view of the back surface 840 of the cartridge housing 802 of a single-use cartridge system 800. The cartridge housing 802 includes a top portion 804 and a bottom portion 810. The male keying portions (824, 826) are shown extending from the bottom portion 810 of the cartridge housing 802.

FIG. 8D illustrates a view of the front surface 850 of the cartridge housing 802 of a single-use cartridge system 800. The male keying portions (824, 826) are shown extending from the bottom portion 810 of the cartridge housing 802.

FIG. 8E illustrates a view of one side surface 860 of the cartridge housing 802 of a single-use cartridge system 800, the opposing side being a mirror image.

FIG. 8F illustrates a cross-section view downward of a single-use cartridge system 800 along the horizontal line of FIG. 8E. The cartridge system 800 includes a detection region 831 that accommodates or is otherwise adapted to receive a chip 832 and flow cell wafer 888. The single-use cartridge system 800 includes a microfluidic system 870 for communicating or otherwise providing a means for a test sample to move through the cartridge system 800 and allow for detection and analysis of one or more analytes. The microfluidic system 870 includes an injection port 812 for introduction of a test sample. The injection port may 812 optionally include a check valve 872. The microfluidic system 870 further includes a first microchannel section 874 having a first end 876 attached in communication with the injection port check valve 872 and a second end 878 in communication with a mixing bladder 880. A filter 877 may be located anywhere within the first microchannel section 874. The microfluidic system 870 also includes a vent port 820 within the first microchannel section 874 between the first end 876 and second end 878. The mixing bladder 880 includes a temperature control means 881 in the form of a metal coil wrapped around the mixing bladder 880 such that the temperature control means 881 is heated upon introduction of an electric current.

The microfluidic system 870 further includes second microchannel section 882 having a first end 884 attached in communication with the mixing bladder 880 and a second end 886 attached in communication with a flow cell wafer 888 having at least one detection microchannel 834.

The microfluidic system 870 further includes third microchannel section 890 having a first end 892 attached in communication with at least one detection microchannel 834 and a second end 894 in communication back to the mixing bladder 880 so as to form a closed loop.

The microfluidic system 870 further includes at least one micropump 898. The micropump 898, as illustrated, is a piezoelectric pump that overlays or otherwise engages or touches one or more of the first microchannel section 874, second microchannel section 882, third microchannel section 890 and mixing bladder 880. The micropump 898 manipulates the communication of test sample throughout the microfluidic system 870.

The single-use cartridge system 800 may further include a transmission component 897 as provided herein. The single-use cartridge system 800 may further include a location means 899 as provided herein.

An exemplary embodiment of a multiple-use cartridge system 900 is illustrated in FIGS. 9A-F.

A top view of an embodiment of a multi-use cartridge system 900 is provided in FIG. 9A. The cartridge system 900 includes a cartridge housing 902 as described herein. The housing 902 includes a top portion 904 (see FIG. 9C) having a top surface 905. As illustrated, the top surface 905 includes four top through holes 908A. The top through holes 908A are adapted (e.g., threaded) to receive a removable fastening means (not shown) for securing the top portion 904 to a bottom portion 910 (see FIG. 9C). The top surface also includes two sealing holes 908B that allow for sealing of the chip 936 to the cartridge housing 902.

The cartridge housing 902 further includes an electronic communication means 916 located on a second external surface 918 that is on a different horizontal plane from the top surface 905. The electronic communication means 916 as illustrated includes a plurality of metal contacts. The top surface 905 also includes two port seals 919 and two seal plugs (924, 926).

FIG. 9B illustrates a view of the bottom surface 923 of a multiple-use cartridge system 900. The bottom surface 923 includes a first male key portion 920 and a second male key portion 922. The male keying portions (920, 922) engage with a corresponding rail portion (425-See FIGS. 5A and 5B) located in the interferometric system. The bottom surface 923 further defines a first detent 928 and a second detent 930. The detents (928, 930) engage with or otherwise receive a corresponding first raised surface and a second raised surface (421A, 421B) inside the cartridge recess 430 of the optical assembly 400 (see FIGS. 5A, 5B and 5C). When engaged with the first detent 928 and second detent 930, the first raised surface and second raised surface (421A, 421B) aid in securing the cartridge system 900 within the cartridge recess 430.

The bottom surface further includes bottom through holes 908C that align and correspond to the four top through holes 908A. The bottom through holes 908C may be adapted (e.g., threaded) to receive a removable fastening means (not shown) for securing the top portion 904 to a bottom portion 910 (see FIG. 9C).

The bottom surface 923 further defines a light inlet slot 934. The light inlet slot 934 allows for a light signal to enter the cartridge system 900. Particularly, the light inlet slot 934 allows for a light signal to enter the chip 936 and for the light signal to move through any waveguides in the chip 936 while interacting with analytes in the at least one detection microchannel 994 (see FIG. 9F) before the light signal is deflected by one or more gratings (not shown) down to the detector unit 406 (see FIG. 6).

FIG. 9C illustrates a view of the back surface 940 of one embodiment of a multiple-use cartridge system 900. The housing includes a top portion 904 that is optionally removable from a bottom portion 910. The male keying portions (920, 922) are shown extending from the bottom portion 910 of the cartridge housing 902.

FIG. 9D illustrates a view of the front surface 950 of one embodiment of a multiple-use cartridge system 900. FIG. 9E illustrates view of one side surface 960 of one embodiment of a single-use cartridge system 900, the opposite side being a mirror image.

FIG. 9F illustrates a cross-section view downward of a multiple-use cartridge system 900 along the horizontal line of FIG. 9E. The cartridge system 900 may contain a storage means 907 as provided herein positioned within the cartridge housing 902. The multiple-use cartridge system 900 includes a microfluidic system 970 for communicating or otherwise providing a means for a test sample to move through the cartridge system 900 and allow for detection and analysis of one or more analytes. An ingress port 972 is located on a front surface 950 (see FIG. 9D) of the multiple-use cartridge system 900. The ingress port 972 is in communication with a first microchannel section 974 having a first end 976 attached in communication with an ingress port check valve 973 and a second end 978 in communication with second microchannel section 979. A filter 977 may be located anywhere within the first microchannel section 974. A sample electrode 980 and reference electrode 982 are in contact with the second microchannel section 979. Impedance may be measured between the sample electrode 980 and reference electrode 982 to confirm the presence of test sample.

A valve test structure connection 984 is in communication with any test sample in the microfluidic system 970. The valve test structure connection 984 may be fabricated from nitinol shape memory alloy and aids in the movement of test sample into the cartridge system 900.

The second microchannel section 979 includes a first end 988 in communication with the first microchannel section 974 and a second end 990 in communication with a flow cell 992 having at least one detection microchannel 994. The cartridge system 900 includes a detection region 993 that accommodates or is otherwise adapted to receive the chip 936 and flow cell 992. The chip 936 is substantially transparent and allows the light signal to enter, interact with one or more waveguides channels (not shown; see e.g., part 316 of FIG. 3C) and allow for binding of analyte flowing within the at least one detection microchannel 994 within the flow cell 992.

The detection microchannel 994 is in communication with a first end 996 of a third microchannel section 998. The third microchannel section 998 includes a flow electrode 1000 to approximate flow rate and is correlated with measured impedance. The third microchannel section 998 includes a second end 1002 in communication with the first end 1004 of a fourth microchannel 1006. The fourth microchannel 1006 includes a second end 1008 in communication with a check valve 1010 which, in turn, is in communication with an egress port 1012 (see also FIG. 9D). The sample electrode 980, reference electrode 982, and flow electrode 1000 are each fabricated from inert nitinol or other corrosion-resistant conductive material.

The multiple-use cartridge system 900 may further include a transmission component 1014 as provided herein. The multiple-use cartridge system 900 may further include a location means 1016 as provided herein.

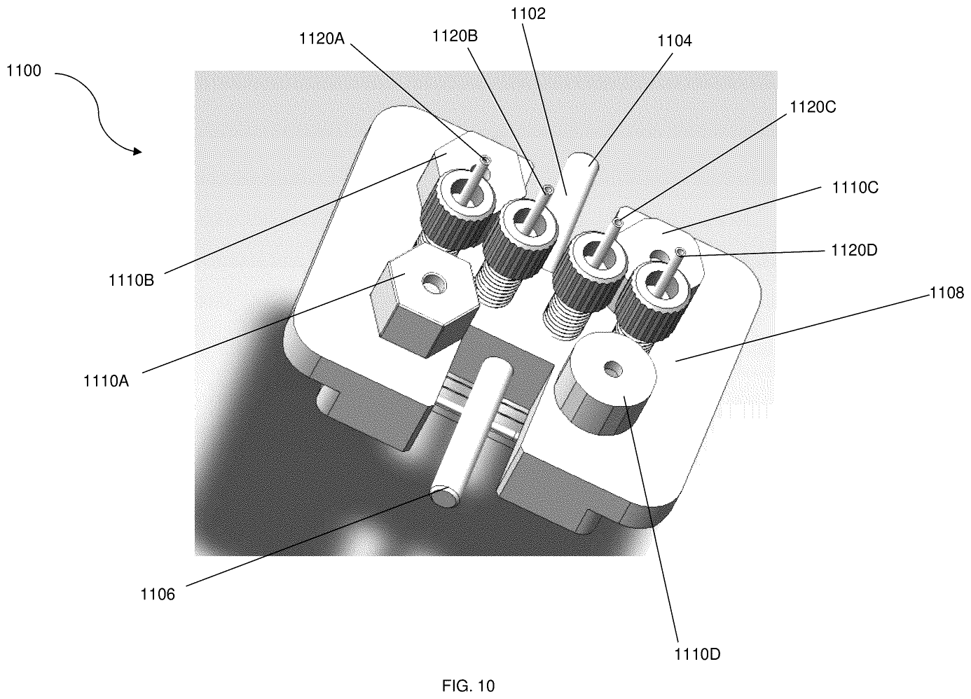

An exemplary embodiment of an alternative single-use cartridge system 1100 is illustrated in FIG. 10. According to the illustrated embodiment, the cartridge system 1100 includes a connection mechanism 1102 (or snap-in rod) having opposing ends (1104, 1106) extending from the housing 1108. The connection mechanism 1102 aids in securing and interfacing the cartridge system 1100 with an interferometric system. Rising from the housing 1108, are injection ports 1110 A-D and outlet ports 1120 A-D. The injection ports 1110 A-D may be utilized for introducing a test sample, buffer or a test sample. The cartridge system includes four independent detection microchannel ports that are independently in communication with a corresponding detection microchannel (not shown) within a flow cell (not shown). Buffer may be pre-loaded in the flow cell. Any test sample waste may be collected from the outlet ports 1120 A-D.

### Fluoro-Containing Substance Applications

By being mobile and utilized near the point where one or more analyte needs to be measured, a user may receive results in an efficient manner and any care or remedial measure decisions may be implemented immediately. The interferometric systems provided herein provide a major technical advancement to detect, quantify and even track various fluoro-containing substances. The systems provided herein may also provide a means to indicate and otherwise aid in the control of the processing, storage, and movement of fluoro-containing substances. The systems provided herein also provide a means to assess the presence of fluoro-containing substances in or on a variety of environments.

According to a particular embodiment, the systems provided herein may be utilized to detect and quantify levels of a perfluoroalkyl and polyfluoroalkyl substances (PFAS). Exemplary PFAS include, but are not limited to, perfluorooctanoic acid (PFOA), perfluorooctane sulfonic acid (PFOS), perfluorononanoic acid (PFNA), hexafluoropropylene oxide dimer acid (HFPO-DA, commonly known as GenX Chemicals), perfluorohexane sulfonic acid (PFHxS), perfluorobutanoic acid (PFBA), perfluorodecanoic acid (PFDA) and perfluorobutane sulfonic acid (PFBS).

According to one embodiment, a fluid source of analytes includes an industrial or commercial vessel adapted to store, process, or carry one or more chemicals that may contain fluoro-containing substances. Such a vessel may be located within or around a shipping container that stores and transports a fluid chemical. The shipping container may be located on a truck, train, or other means of transportation. The shipping container may also be located on or around shipping dock.

According to a particular embodiment, the systems and methods provided herein may be utilized to detect and quantify levels of a fluoro-containing substances in an industrial environment such as in a chemical processing or chemical manufacturing facility. By providing detection and quantification data in an efficient manner within the production environment, exposure to fluoro-containing substances may be monitored, adjusted and otherwise controlled. According to such an embodiment, the system will detect and quantify one or more fluoro-containing substances at the parts per million (ppm), parts per billion (ppb) or parts per trillion (ppt) level.

According to a particular embodiment, the systems and methods provided herein may be utilized to detect, quantify or otherwise monitor levels of a fluoro-containing substances in a variety of other applications in various environments. Such applications include waste management, national defense, metal coating, plastics/resins, mining and refining, airports, petroleum extraction and refining, paints/coatings, metal machinery manufacturing, paper mills, packaging production, disposable plate production, electronics, oil/gas, printing, textiles/leathers, cleaning products manufacturing, food production, glass products, cement manufacturing, fire departments (fire suppression at home or on truck), home accessories/building products (e.g., carpet, furniture), water treatment (municipal, well, bottled) and airport fire suppression foam cleanup.

According to one particular embodiment, the interferometric system provided herein may be utilized in connection with or otherwise equipped to a mobile vehicle. Suitable mobile vehicles include, but are not limited to, unmanned aerial vehicles (UAV), unmanned ground vehicles (UGV), drones, manned aircraft, and manned vehicles.

According to one particular embodiment, the interferometric system provided herein may be utilized in connection with or otherwise equipped to a water supply system to continuously monitor (or batch monitor) the water for fluoro-containing substances. According to one particular embodiment, the interferometric system provided herein may be connected to a water faucet in a variety of locations such as in a home, laboratory or industrial setting.

### Methods of Detection and Quantification

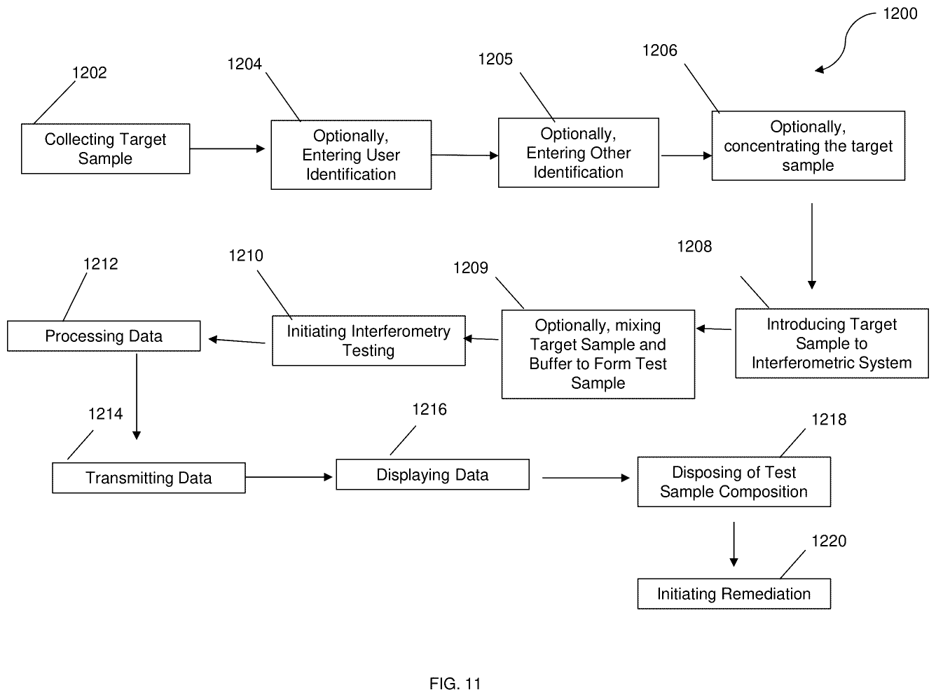

FIG. 11 illustrates a method 1200 of detecting and quantifying the level of analyte in a target or test sample. The method includes the step of collecting 1202 or otherwise obtaining a target sample having one or more analytes. In different embodiments, the target sample may be taken from the appropriate target depending on the location and environment.

According to one embodiment, the method further includes the optional step of entering 1204 a user identifier (ID) in the system. Additionally, an identification number associated with the sample, analyte or interest or a combination thereof may be entered. The cartridge system utilized may be equipped with a label or sticker carrying identifying information.

According to one embodiment, the method further includes the optional step of entering other information 1205. The label or sticker may include a QR code including such information The label or sticker may be removed prior to use. Identifying information may include metadata such as time, GPS data, or other data generated by the interferometric system.

According to one embodiment, the method further includes the step of concentrating 1206 the test sample. According to one embodiment, the step of concentrating is carried out by solid-solid extraction/solid phase extraction (SPE), microbeads, or other suitable means for concentrating, purifying or otherwise preparing the test sample for detection and quantification.

According to one embodiment, the method further includes the step of introducing the target sample to the interferometric system 1208. According to one embodiment, target sample is introduced to the cartridge by a separate device such as a syringe or pump. According to one embodiment, target sample is introduced by an injection device. According to one embodiment, the injection device may be permanently attached to the cartridge system. According to one embodiment, the injection device is a pipette. According to one embodiment, the injection device is a syringe. According to one embodiment, the injection device is a lance, pipette or capillary tube. When utilizing a multiple-use cartridge system, the cartridge system may be fitted to a tube or other transfer mechanism to allow the sample to be continuously taken from a large amount of fluid that is being monitored.

According to one embodiment, the method further includes the step of mixing 1209 the target sample with a buffer solution to form a test sample. According to one embodiment, the buffer solution is aqueous based. In a multiple-use cartridge system, such a step may occur prior to the test sample being introduced to the cartridge system. In a single-use cartridge system, such a step may occur in the mixing bladder with the assistance of a pump.

The method of detecting and quantifying the level of analyte in a sample includes initiating waveguide interferometry 1210 on the test sample. Such a step may include initiating movement of the light signal through the cartridge system as provided herein and receiving the light signal within the detector unit. Any changes in an interference pattern are representative of analyte in the test sample. Particularly, such changes in an interference pattern generate data related to one or more analyte in the test sample. According to one embodiment, the step of initiating 1210 waveguide interferometry on the test sample includes the step of correlating data from the phase shift with calibration data to obtain data related to analyte identity, analyte concentration, or a combination thereof.

According to one embodiment, the method further includes the step of processing 1212 any data resulting from changes in the interference pattern. Such changes in interference pattern may be processed and otherwise translated to indicate the presence and amount of an analyte in a test sample. Processing may be assisted by software, processing units, processor, servers, or other component suitable for processing. The step of processing data may further include storing such data in storage means as provided herein.

According to one embodiment, the method further includes the step of transmitting a data signal 1214. The signal may result in the displaying of data on the system 1216. The step of transmitting data may include displaying the analyte levels via projecting any real time data on a screen as described herein. The step of transmitting data may include transmitting any obtained data to a mobile phone, smart phone, tablet, computer, laptop, watch or other wireless device. The data may also be sent to a device at a remote destination. The remote destination device may be a locally operated mobile or portable device, such as a smart phone, tablet device, pad, or laptop computer. The destination may also be smart phone, pad, computer, cloud device, or server. In other embodiments, the remote destination may be a stand-alone or networked computer, cloud device, or server accessible via a local portable device.

The method may optionally include the step of disposing of the test sample 1218 per legal requirements. Such legal requirements assure that any sample still containing unacceptable levels of pathological contamination are disposed of properly so as not to cause harm to a user or the environment.

According to one embodiment, the method optionally includes the step of initiating 1220 a cleaning or remedial countermeasure against any analyte detected. Such remedial measure may include introducing cleaning chemicals to a particular environment where fluoro-containing substances are located. The remedial measures may work to kill or otherwise neutralize any unwanted analyte present in the environment where a sample was taken.

According to a particular embodiment, a method of detecting and quantifying the level of analyte in a target sample is provided that includes the steps of:
collecting a target sample containing one or more analytes;
optionally entering an identification associated with the target sample;
optionally, concentrating the target sample;
optionally, mixing the target sample with a buffer solution to form a test sample composition;
introducing the target sample to an interferometer;
initiating waveguide interferometry on the target sample;
processing any data resulting from the waveguide interferometry; and
optionally, transmitting any data resulting from the waveguide interferometry. According to one embodiment, the interferometric device utilized in the methods provided herein may include an interferometric chip that, in turn, includes one or more waveguide channels having a sensing layer composition thereon. According to one embodiment, the sensing layer composition includes at least one polymeric receptor and at least one amino silane. According to one embodiment, the sensing layer composition is adapted to bind or otherwise be selectively disturbed by one or more analytes within the target sample.

Although the present specification describes components and functions that may be implemented in particular embodiments with reference to particular standards and protocols, the invention is not limited to such standards and protocols. For example, standards for Internet and other packet switched network transmission (e.g., TCP/IP, UDP/IP, HTML, HTTP) represent examples of the state of the art. Such standards are periodically superseded by faster or more efficient equivalents having essentially the same functions. Accordingly, replacement standards and protocols having the same or similar functions as those disclosed herein are considered equivalents thereof.

### Method of Test Sample Composition Preparation

According to one aspect of the disclosure, a method for preparing a test sample composition is provided. In certain embodiments, the method is configured for implementation in the field, followed by a method of detecting and quantifying one or more analytes using an interferometric system. The method enables preparation of a test sample composition from a raw or environmental target sample for improved analyte detection sensitivity and compatibility with the sensing platform such as the interferometric system described herein. According to one embodiment, to enable rapid, on-site PFAS detection, an interferometric system is utilized that incorporates an integrated sample extraction and concentration module to perform the method for preparing a test sample composition.

In one embodiment, the method for preparing a test sample composition includes a step of performing extraction of the target sample using solid-solid extraction, solid-phase extraction (SPE), microbeads, or any other suitable field compatible material or technique capable of isolating or enriching target analytes of interest. The extraction step may be adapted for use in portable or field-based settings and may be designed for ease of use with simplified protocols, minimal sample handling, and reduced solvent requirements. The extraction step may be utilized with reduced solvent use and simplified handling steps suitable for non-laboratory personnel.

According to one embodiment, the method for preparing a test sample composition includes a target sample concentration step. According to one embodiment, the method of concentrating the target sample includes a buffer adjustment or conditioning step. This step is configured to tune the test sample to conform to the buffer conditions required by the optical interferometric sensing system while simultaneously reducing matrix interferences that may affect signal quality. The buffer may be selected to support optimal binding of analytes to the sensing layer composition and maintain the stability of the interferometric signal. According to one embodiment, the method of concentrating the target sample includes a controlled evaporation step to reduce final sample volume thereby selectively enriching the concentration of analytes, such as perfluoroalkyl and polyfluoroalkyl substances (PFAS). By increasing the local concentration of PFAS analytes in the target sample, this step facilitates detection within the dynamic range of the interferometric sensor, thereby lowering the effective detection limit and improving measurement precision, reproducibility, and confidence.

According to one embodiment, the evaporation step may be achieved by heating, agitation, or blow-down methods. According to one embodiment, the concentrated target sample is then resuspended in a buffer solution that is sensor-compatible to bring analyte levels within the dynamic detection range of the sensor. The concentration step improves detection limits to the parts-per-trillion (ppt) range and minimizes matrix interference.

According to one embodiment, the method for preparing a test sample composition includes a step of matching buffer to further reduce background interference and improve detection accuracy. According to one embodiment, during the extraction step, a flow-through (post-extraction aqueous buffer phase) is collected and reused as a baseline buffer for the subsequent sensing measurement.

According to one embodiment, the method for preparing a test sample composition includes a step of collecting any aqueous buffer phase obtained during the extraction step and reusing the collected buffer as a recycled baseline buffer in the interferometric detection step, wherein the recycled baseline buffer contains no detectable PFAS and closely matches any buffer of the resuspended test sample composition, thereby reducing background interference and improving detection accuracy. By utilizing baseline, recycled buffer, the opportunity for buffer mismatches is reduced while enhancing measurement reliability. Additionally, this buffer recycling approach minimizes waste generation and lowers the volume of consumables required in field deployment.

Although specific embodiments of the present disclosure are herein illustrated and described in detail, the disclosure is not limited thereto. The above detailed descriptions are provided as exemplary of the present disclosure and should not be construed as constituting any limitation of the disclosure. Modifications will be apparent to those skilled in the art, and all modifications that do not depart from the spirit of the disclosure are intended to be included with the scope of the appended claims.

### STATEMENTS OF THE DISCLOSURE

Statement 1. A sensing layer composition comprising:
   at least one polymeric receptor comprising a carboxylic acid-substituted cyclodextrin derivative; and
   at least one amino silane,
   wherein the sensing layer composition is adapted to be adhered to at least one side of one or more waveguide channels in/on a waveguide chip of an interferometric system,
   wherein the sensing layer composition is adapted to bind or otherwise be selectively disturbed by one or more analytes, and
   wherein the sensing layer composition is configured to bind or otherwise be selectively disturbed by one or more analytes in a test sample at a flow rate through the interferometric system of up to about 5.0 mL/min.
Statement 2. The sensing layer composition of statement 1, wherein the interferometric system is an optical interferometric system.
Statement 3. The sensing layer composition of statement 1, wherein the carboxylic acid-substituted cyclodextrin derivative is covalently linked to the amino silane via EDC/NHS-mediated coupling to form a receptor-functionalized film.
Statement 4. The sensing layer composition of statement 1, wherein the at least one polymeric receptor is formed from cyclodextrin, 1-Ethyl-3-(3-dimethylaminopropyl) carbodiimide (EDC), N-Hydroxy succinimide (NHS), or a combination thereof.
Statement 5. The sensing layer composition of statement 1, wherein the one or more analytes that may be detected include at least one fluoro-containing substance.
Statement 6. The sensing layer composition of statement 1, wherein the one or more analytes include, but are not limited to, perfluoroalkyl and polyfluoroalkyl substances (PFAS).
Statement 7. The sensing layer composition of statement 6, wherein the PFAS is selected from the group consisting of perfluorooctanoic acid (PFOA), perfluorooctane sulfonic acid (PFOS), perfluorononanoic acid (PFNA), hexafluoropropylene oxide dimer acid (HFPO-DA), perfluorohexane sulfonic acid (PFHxS), and perfluorobutane sulfonic acid (PFBS).
Statement 8. The sensing layer composition of statement 6, wherein the binding of the PFAS alters the local refractive index of the waveguide channel, resulting in a detectable interferometric response.
Statement 9. The sensing layer composition of statement 1, wherein the at least one amino silane includes 3-aminopropyltriethoxysilane (APTES).
Statement 10. The sensing layer composition of statement 1, wherein the amino silane is adapted to covalently bind to a silicon dioxide surface of the waveguide channel.
Statement 11. The sensing layer composition of statement 1, wherein the sensing layer composition is hydrophilic and configured to selectively bind one or more perfluoroalkyl and polyfluoroalkyl substances (PFAS) through a combination of hydrophobic and ionic interactions.
Statement 12. The sensing layer composition of statement 1, wherein the polymeric receptor is formed by reacting excess EDC and NHS with the carboxylic acid-substituted cyclodextrin in aqueous solution.
Statement 13. The sensing layer composition of statement 1, wherein the sensing layer is deposited on the waveguide chip by spin coating and forms a continuous film.
Statement 14. The sensing layer composition of statement 1, wherein the composition comprises about 0.1% w/w to about 50% w/w of the amino silane and about 0.1% w/w to about 50% w/w of the polymeric receptor, based on the total weight of the sensing layer composition.
Statement 15. The sensing layer composition of statement 1, wherein the cyclodextrin derivative is a α, β or γ-cyclodextrin substituted at the primary hydroxyl position with a carboxylic acid moiety to promote ionic interaction with PFAS compounds.
Statement 16. The sensing layer composition of statement 1, wherein the film exhibits reversible binding to fluoro-containing analytes such that re-equilibration with buffer regenerates the surface for reuse within 30 minutes.
Statement 17. The sensing layer composition of statement 1, wherein the film maintains stability in a water-based buffer comprising up to 5% v/v ethanol or methanol and is free of signal degradation over a continuous 4-hour measurement period.
Statement 18. The sensing layer composition of statement 1, wherein the polymeric receptor layer provides a dynamic response curve to PFAS concentrations ranging from 5 parts per billion to 500 parts per billion with a coefficient of variation less than 10%.
Statement 19. The sensing layer composition of statement 1, wherein the sensing layer composition is applied to both sensing and reference waveguide channels in a differential mode to enable internal correction for environmental noise.
Statement 20. A method for detecting one or more analytes in a test sample composition using an interferometric sensing system, the method comprising:
   providing a waveguide chip comprising one or more waveguide channels, each channel having disposed thereon a sensing layer composition, such as that in statement 1, comprising at least one polymeric receptor comprising a carboxylic acid-substituted cyclodextrin derivative and at least one amino silane;
   introducing the test sample composition to the waveguide chip under conditions sufficient to allow selective binding of the one or more analytes to the sensing layer composition; and
   detecting a change in the optical interference signal resulting from binding of the one or more analytes to the sensing layer composition.
Statement 21. The method of statement 20, further comprising rinsing the waveguide chip with a buffer solution to regenerate the sensing layer composition, wherein the sensing layer composition retains analyte binding capability after regeneration.
Statement 22. The method of statement 20, wherein the one or more analytes include perfluoroalkyl and polyfluoroalkyl substances (PFAS), and the binding of the PFAS to the sensing layer composition causes a change in the local refractive index sufficient to generate a detectable interferometric response.
Statement 23. The method of statement 20, wherein the interferometric sensing system is configured as a portable or field-deployable device.
Statement 24. The method of statement 20, wherein the sensing layer composition is applied to both a sensing waveguide channel and a reference waveguide channel, and the change in the optical interference signal is determined using differential analysis between the sensing and reference channels.
Statement 25. A method of detecting and quantifying one or more perfluoroalkyl or polyfluoroalkyl substances (PFAS) in a test sample composition, the method comprising:
   obtaining a water sample from an environmental source selected from semiconductor wastewater, groundwater, pore water, or drinking water, wherein the water sample is optionally pre-treated using surface active foam fractionation (SAFF);
   filtering the water sample to remove particulates;
   extracting and concentrating PFAS from the filtered sample using solid-phase extraction (SPE);
   reconstituting the extracted analytes in a buffer solution to form a test sample composition;
   introducing the test sample composition to a waveguide chip comprising a sensing layer composition disposed on at least one waveguide channel, wherein the sensing layer composition comprises a polymeric receptor including a carboxylic acid-substituted cyclodextrin derivative and at least one amino silane; and
   detecting a change in optical interference signal resulting from binding of PFAS to the sensing layer composition, thereby quantifying the PFAS concentration in the sample
Statement 26. The method of statement 25, further comprising collecting any aqueous buffer phase obtained during the extraction step as a flow-through and reusing the collected buffer as a baseline running buffer in the interferometric detection step, wherein the recycled buffer contains no detectable PFAS and closely matches any buffer of the resuspended test sample composition, thereby reducing background interference and improving detection accuracy.
Statement 27. A method for preparing a test sample composition for detection of one or more analytes using an interferometric sensing system as provided herein, the method comprising:
   collecting a target sample from an environmental source suspected of containing one or more analytes;
   performing an extraction step on the target sample using a field-compatible extraction material selected from solid-solid extraction media, solid-phase extraction (SPE), or microbeads to isolate the one or more analytes;
   concentrating the extracted analytes by reducing sample volume using a method selected from heating, agitation, or blow-down evaporation;
   resuspending the concentrated analytes in a buffer solution that is compatible with the interferometric sensing system; and
   preparing a test sample composition by conditioning the buffer to reduce matrix interferences and improve compatibility with a sensing layer composition on a waveguide chip.
Statement 28. The method of statement 27, further comprising collecting an aqueous buffer phase during a sample preparation step and reusing the collected buffer as a recycled baseline buffer in the interferometric detection step, wherein the recycled baseline buffer contains no detectable PFAS and closely matches the buffer used to resuspend the test sample composition, thereby reducing background interference and improving detection accuracy.
Statement 29. The method of statement 27, wherein the target sample is selected from groundwater, surface water, pore water, drinking water, or semiconductor wastewater.
Statement 30. The method of statement 27, wherein the buffer solution in the resuspension step is selected to maintain binding affinity between a sensing layer composition comprising a carboxylic acid-substituted cyclodextrin derivative and the one or more analytes.
Statement 31. The method of statement 27, wherein the analytes comprise perfluoroalkyl or polyfluoroalkyl substances (PFAS), and the concentration step enriches the analytes into a detectable range of between 1 part per trillion (ppt) and 500 parts per billion (ppb).
Statement 32. The method of statement 27, wherein the extraction, concentration, and detection steps are performed using an integrated interferometric sensing platform comprising a modular housing, a waveguide chip with a sensing layer composition, a fluidic delivery system, and a control interface.
Statement 33. The method of statement 27, wherein the method is performed using a portable, battery-powered sensing unit configured for use outside a laboratory setting, and the unit comprises onboard heating or blow-down components for sample evaporation and integrated buffer conditioning.
Statement 34. The method of statement 27, wherein the extraction and buffer preparation steps are conducted using pre-loaded cartridges or consumables adapted for simplified handling by non-technical personnel in field conditions.
Statement 35. The method of statement 27, wherein the extraction step is conducted using less than 10 milliliters of organic solvent per sample, thereby reducing chemical waste and enhancing field safety.
Statement 36. The method of statement 27, wherein the integrated interferometric sensing platform includes automated fluid handling components to perform at least one of the extraction, buffer exchange, or analyte detection steps without manual intervention.
Statement 37. The method of statement 27, wherein the sensing layer composition is configured to bind or otherwise be selectively disturbed by one or more analytes at a flow rate through the interferometric system of up to about 5.0 mL/min.
Statement 38. The method of any of statements 20-37, wherein detection and, optionally, quantification are performed on an optical interferometric system as described herein.
Statement 39. The method of any of statements 20-38, wherein the polymeric receptor is 3-aminopropyltriethoxysilane (APTES) and the amino silane is succinyl β-cyclodextrin.

### EXAMPLE 1

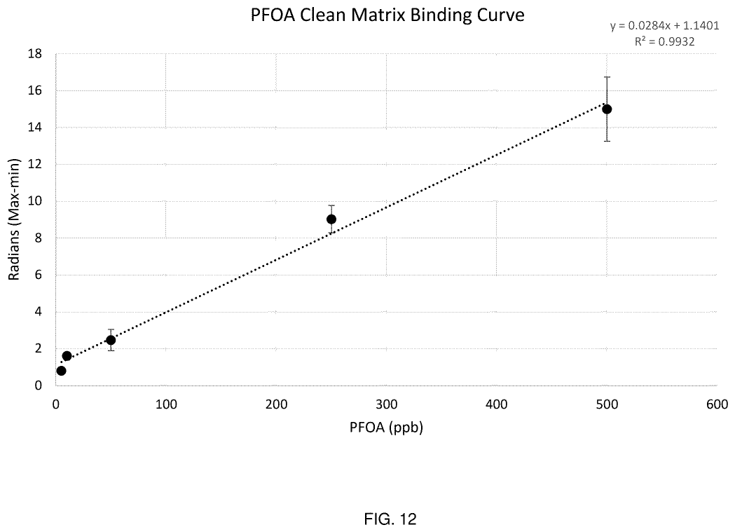

Testing was conducted in an interferometric system as provided herein having a sensing layer composition including a cyclodextrin polymer sensing receptor functionalized to the surface of the waveguide channels via amnio silane immobilization on at least one waveguide channel. Perfluorooctanoic acid (PFOA) stock solutions and samples were prepared at the following concentrations: 5 ppb, 25 ppb, 10 ppb, 50 ppb, 100 ppb, 250 ppb, and 500 ppb. A blank (0 ppb) sample was also made and used to run as a negative control. The PFOA test sample compositions were prepared in buffer containing 100% v/v deionized water. The sensing layer composition including a cyclodextrin sensing receptor was equilibrated on the waveguide channels with running buffer (100% DI water) until stabilized. Test sample compositions were added over the sensing layer composition (sample introduced at 0 seconds on graph). Test sample compositions hit the sensing layer composition after about 150 seconds from sample introduction where binding began, and a response was observed. At 1000 seconds, running buffer was introduced again and PFOA sample was no longer flowed over the sensing surface. Total response during sample time was calculated by the maximum response value with the minimum response value from 200-500 seconds. The results are illustrated in the graph of FIG. 12.

During the 1000 second sample introduction, it was observed that a user can differentiate between PFOA samples and a blank sample (0 ppb PFOA) as well as differentiate the total response at a given PFOA concentration as low as 5 ppb and as high as 500 ppb. Concentrations higher than 500 ppb were not attempted in this Example 1.

### EXAMPLE 2

A method of sample composition preparation followed by detection and quantification was validated against the industry standard method (LC/MS/MS 1633) utilizing one embodiment of an interferometric system as described herein. Testing was conducted in the interferometric system which included a sensing layer composition having a cyclodextrin polymer sensing receptor functionalized via an amino silane immobilization to the surface of the waveguide channels. Environmental target samples were received from industry partners and spiked with perfluorooctanoic acid (PFOA) to a known concentration of 50 parts per billion. Sample sources included semiconductor wastewater, groundwater, drinking water, pore water, and influent and effluent samples associated with surface-active foam fractionation (SAFF) treatment systems. Each target sample was treated through sample preparation methods as provided herein and run through an interferometric system described herein to obtain a result.

Each target sample was first subjected to a preliminary filtration step to remove particulates. The filtration was performed using a glass fiber filter appropriate for field and laboratory use.

Following filtration, the filtered target sample was subject to a step of analyte extraction. Specifically, the filtered target sample was introduced to a solid phase extraction (SPE) cartridge thereby extracting the analyte into a methanol solution. The methanol solution was then evaporated off and analyte was resuspended in a compatible buffer without interfering with matrices (DI water) to form a test sample composition. The sensing layer composition including a cyclodextrin sensing receptor was equilibrated on the waveguide channels with running buffer (100% DI water) until stabilized. This process was performed on each of the samples to form respective test sample compositions.

Test sample compositions were then introduced over the sensing layer composition at a flow rate of about 0.4 mL/min. Samples hit the sensing layer composition after about 150 seconds from test sample composition introduction where binding began, and a response was observed. At 1000 seconds, running buffer was introduced again and target samples were no longer flowed over the sensing surface. Total response during sample time was calculated by the maximum response value with the minimum response value from 200 to 500 seconds. The results are illustrated in Table 1. The entire process from test sample composition preparation to detection was conducted in less than 4 hours.

The experimental data presented herein demonstrate that the methods of sample composition preparation followed by detection and quantification are capable of accurately detecting and quantifying perfluorinated analytes across a range of environmentally and industrially relevant samples. The results obtained using the interferometric sensing system, which incorporates the disclosed sensing layer composition which utilize a polymeric receptor and amino silane immobilization on waveguide channel surfaces, exhibit close correlation with data generated using the industry standard (LC/MS/MS Method 1633) while also being field deployable. Across all tested samples, including complex samples such as semiconductor wastewater and groundwater, the interferometric detection method provided quantitative outputs within acceptable variance of the validated reference method. Accordingly, these results support the conclusion that the disclosed methods enable rapid, reliable, and field-deployable detection and quantification of PFAS compounds with analytical accuracy sufficient for practical environmental monitoring applications.

TABLE 1

| | Detected and | | Detected and | | Quantified PFAS | |
|---|---|---|---|---|---|---|
| | Quantified PFAS | | Quantified PFAS | | validated by | |
| Sample | Level (ppb) | | Average (ppb) | | LC/MS/MS | |
| Semiconductor | 226.3 | ppb | 192.6 | ppb | 195.2 | ppb |
| wastewater | 183.6 | ppb | | | | |
| | 168.1 | ppb | | | | |
| Groundwater | 39.9 | ppb | 51.3 | ppb | 84.8 | ppb |
| | 33.6 | ppb | | | | |
| | 80.4 | ppb | | | | |
| Pore water | 38.2 | ppb | 62.6 | ppb | 64.8 | ppb |
| “Water | 86.9 | ppb | | | | |
| between soil, | | | | | | |
| sediment or | | | | | | |
| rock” | | | | | | |
| Influent | 20.1 | ppb | 20.1 | ppb | 75.2 | ppb |
| Groundwater | 20.2 | ppb | | | | |
| (SAFF) | | | | | | |
| Groundwater | Not Detected - | | Not Detected - | | .451 | ppb |
| After | Under Dynamic | | Under Dynamic | | | |
| Treatment | Range Limit of | | Range Limit of | | | |
| (SAFF) | Detection | | Detection | | | |
| Groundwater | Under Limit of | | 13.56 | ppb | 38.1 | ppb |
| before | Detection | | | | | |
| secondary | 34.9 | ppb | | | | |
| treatment | 27.6 | ppb | | | | |
| (SAFF) | | | | | | |
| Drinking | 210.9 | ppb | 178.4 | ppb | 247.8 | ppb |
| Water | 145.9 | ppb | | | | |

We claim:

1. A sensing layer composition comprising:
at least one polymeric receptor comprising a carboxylic acid-substituted cyclodextrin derivative; and
at least one amino silane,
wherein the sensing layer composition is adapted to be adhered to at least one side of one or more waveguide channels in/on a waveguide chip of an interferometric system,
wherein the sensing layer composition is adapted to bind or otherwise be selectively disturbed by one or more analytes, and
wherein the sensing layer composition is configured to bind or otherwise be selectively disturbed by one or more analytes in a test sample at a flow rate through the interferometric system of up to about 5.0 mL/min.

2. The sensing layer composition of claim 1, wherein the interferometric system is an optical interferometric system.

3. The sensing layer composition of claim 1, wherein the carboxylic acid-substituted cyclodextrin derivative is covalently linked to the amino silane via EDC/NHS-mediated coupling to form a receptor-functionalized film.

4. The sensing layer composition of claim 1, wherein the at least one polymeric receptor is formed from cyclodextrin, 1-Ethyl-3-(3-dimethylaminopropyl) carbodiimide (EDC), N-Hydroxy succinimide (NHS), or a combination thereof.

5. The sensing layer composition of claim 1, wherein the one or more analytes that may be detected include at least one fluoro-containing substance.

6. The sensing layer composition of claim 1, wherein the one or more analytes include, but are not limited to, perfluoroalkyl and polyfluoroalkyl substances (PFAS).

7. The sensing layer composition of claim 6, wherein the PFAS is selected from the group consisting of perfluorooctanoic acid (PFOA), perfluorooctane sulfonic acid (PFOS), perfluorononanoic acid (PFNA), hexafluoropropylene oxide dimer acid (HFPO-DA), perfluorohexane sulfonic acid (PFHxS), and perfluorobutane sulfonic acid (PFBS).

8. The sensing layer composition of claim 6, wherein the binding of the PFAS alters the local refractive index of the waveguide channel, resulting in a detectable interferometric response.

9. The sensing layer composition of claim 1, wherein the at least one amino silane includes 3-aminopropyltriethoxysilane (APTES).

10. The sensing layer composition of claim 1, wherein the amino silane is adapted to covalently bind to a silicon dioxide surface of the waveguide channel.

11. The sensing layer composition of claim 1, wherein the sensing layer composition is hydrophilic and configured to selectively bind one or more perfluoroalkyl and polyfluoroalkyl substances (PFAS) through a combination of hydrophobic and ionic interactions.

12. The sensing layer composition of claim 1, wherein the polymeric receptor is formed by reacting excess EDC and NHS with the carboxylic acid-substituted cyclodextrin in aqueous solution.

13. The sensing layer composition of claim 1, wherein the sensing layer is deposited on the waveguide chip by spin coating and forms a continuous film.

14. The sensing layer composition of claim 1, wherein the composition comprises about 0.1% w/w to about 50% w/w of the amino silane and about 0.1% w/w to about 50% w/w of the polymeric receptor, based on the total weight of the sensing layer composition.

15. The sensing layer composition of claim 1, wherein the cyclodextrin derivative is α, β or γ-cyclodextrin substituted at the primary hydroxyl position with a carboxylic acid moiety to promote ionic interaction with PFAS compounds.

16. A method for detecting one or more analytes in a test sample composition using an interferometric sensing system, the method comprising:
providing a waveguide chip comprising one or more waveguide channels, each channel having disposed thereon a sensing layer composition comprising at least one polymeric receptor comprising a carboxylic acid-substituted cyclodextrin derivative and at least one amino silane;
introducing the test sample composition to the waveguide chip under conditions sufficient to allow selective binding of the one or more analytes to the sensing layer composition; and
detecting a change in the optical interference signal resulting from binding of the one or more analytes to the sensing layer composition.

17. The method of claim 16, further comprising rinsing the waveguide chip with a buffer solution to regenerate the sensing layer composition, wherein the sensing layer composition retains binding capability for the one or more analytes after regeneration.

18. The method of claim 16, wherein the one or more analytes include perfluoroalkyl and polyfluoroalkyl substances (PFAS), and the binding of the PFAS to the sensing layer composition causes a change in the local refractive index sufficient to generate a detectable interferometric response.

19. The method of claim 16, further comprising collecting an aqueous buffer phase during a sample preparation step, and reusing the collected buffer as a recycled baseline buffer in the interferometric detection step, wherein the recycled baseline buffer contains no detectable PFAS and closely matches the buffer used to resuspend the test sample composition, thereby reducing background interference and improving detection accuracy.

20. A method of detecting and quantifying one or more perfluoroalkyl or polyfluoroalkyl substances (PFAS) in a test sample composition, the method comprising:
obtaining a water sample from an environmental source selected from semiconductor wastewater, groundwater, pore water, or drinking water, wherein the water sample is optionally pre-treated using surface active foam fractionation (SAFF);
filtering the water sample to remove particulates;
extracting and concentrating PFAS from the filtered sample using solid-phase extraction (SPE);
reconstituting the extracted analytes in a buffer solution to form a test sample composition;
introducing the test sample composition to a waveguide chip comprising a sensing layer composition disposed on at least one waveguide channel, wherein the sensing layer composition comprises a polymeric receptor including a carboxylic acid-substituted cyclodextrin derivative and at least one amino silane; and
detecting a change in optical interference signal resulting from binding of PFAS to the sensing layer composition, thereby quantifying the PFAS concentration in the sample.

21. The method of claim 20, further comprising collecting any aqueous buffer phase obtained during the extraction step, and reusing the collected buffer as a recycled baseline buffer in the interferometric detection step, wherein the recycled baseline buffer contains no detectable PFAS and closely matches any buffer of the resuspended test sample composition, thereby reducing background interference and improving detection accuracy.
